(19) United States
(12) Patent Application Publication
(10) Pub. No.: US 2007/0259397 A1
(43) Pub. Date: Nov. 8, 2007

(54) Enzymes

(75) Inventors: Martinus Julius Beekwilder, Renkum (NL); OIe Sibbesen, Bagsvaerd (DK); Jorn Dalgaard Mikkelsen, Hvidovre (DK); Ingrid Maria Van Der Meer, Wageningen (NL); Robert David Hall, Wageningen (NL); Ingmar Qvist, Norrkouping (SE)

Correspondence Address: STEPTOE & JOHNSON LLP, 1330 CONNECTICUT AVENUE, N.W., WASHINGTON, DC, 20036, US

(73) Assignee: Danisco A/S, Copenhagen (DK)

(21) Appl. No.: 11/658,651

(22) PCT Filed: Jul. 27, 2005
(86) PCT No.: PCT/GB05/02955

§ 371 Date: May 3, 2007

(30) Foreign Application Priority Data
Jul. 28, 2004 (GB) 0416832.4
Aug. 4, 2004 (GB) 0417372.0

(51) Int. Cl. C12P 1/04; C07H 21/04; C12N 1/21; C12N 15/00; C12N 5/06; C12N 9/00

(52) U.S. Cl. 435/68.1; 435/183; 435/243; 435/252.2; 435/252.33; 435/254.11; 435/320.1; 435/325; 435/69.1; 536/23.1

(57) ABSTRACT

The present invention relates to enzymes and processes. In particular, there is described a host cell transformed or transfected with a nucleic acid encoding a plant-derived CCD enzyme.

The present invention relates to enzymes, nucleotide sequences for same and processes using same.

### FIELD OF THE INVENTION

The present invention relates to the field of transformation of bacteria, yeast, fungi, insect, animal and plant cells, seeds, tissues and whole organisms. More specifically, the present invention relates to the integration of recombinant nucleic acids encoding for one or more specific enzymes of the carotenoid biosynthetic and catabolic pathway into suitable host cells which can be used for commercial production of compounds useful for flavouring and fragrance.

### TECHNICAL BACKGROUND AND PRIOR ART

Industries, such as food and beverage, pharmaceuticals, nutraceuticals, soaps and detergents, cosmetics and toiletries, rely on aroma additives to replenish or add flavour to their products. The major source of aroma compounds was originally from the essential oils of plants.

However, plants frequently produce only small amounts of aroma chemicals that are often difficult to isolate. Furthermore, factors including the availability of plants, weather, diseases, labour intensive cultivation, etc., restrict the economic production of large quantities of aroma chemicals.

Accordingly, there is a need for an improved source of such aroma chemicals.

### SUMMARY OF THE INVENTION

In a broad aspect, the present invention relates to organisms, such as microorganisms, which have been modified so as to be capable of producing aroma compounds or precursors thereof.

In one aspect, the present invention relates to organisms, such as microorganisms, which have been modified so as to be capable of producing different carotenoid cleavage compounds.

The present invention is advantageous as it provides for a useful process that is both reliable and efficient for the production of aroma compounds or precursors thereof, in particular carotenoid cleavage compounds, that does not rely solely on chemical synthesis techniques.

In one aspect, the present invention provides a plasmid or a vector system or a transformed or transgenic organism comprising a plant-derived enzyme involved in carotenoid biosynthesis.

In another aspect of the invention there is provided a plasmid or a vector system or a transformed or a transgenic organism comprising a plant-derived carotenoid cleavage dioxygenase (CCD).

A “plant-derived CCD” refers to a CCD gene or enzyme originating from a plant species. Thus, in a preferred embodiment, the nucleotide sequence encoding the CCD which can be used in the host cell of the present invention is obtainable from (though it does not have to be actually obtained from) a plant. In a particularly preferred embodiment, the plant species is selected from Arabidopsis thaliana, Zea mais, tomato or Rubus idaeus (raspberry). Accordingly, in one embodiment there is provided a host cell transformed or transfected with a CCD gene derived from Rubus idaeus.

Other suitable CCD genes from plant species include those derived from Lactuca sativa (lettuce), Vitis vinifera (grape; for example having a sequence set out in accession number CA816346), apple (Malus domesticus) (for example having a sequence set out in accession number CN489783), peach (Prunus persica, for example having a sequence set out in accession number BU041818), almond (for example having a sequence set out in accession number BU574082), crocus sativus, pisum sativum, Phaseolus vulgaris, Brassica napus, Glycine max (soy) Rosa spp., Tomato (for example, having a sequence set out in accession number Z37174), Medicago truncatula (for example, having a sequence set out in accession number BG453-465), poplar (populus), orange (Citrus sinensis) and Ice plant (Mesembryanthemum crystallinum).

Advantageously, and in contrast to mammalian-derived CCD, the plant-derived gene cleaves carotene to generate two moles beta ionone per mole carotene. The mammalian gene cleaves only one beta-ionone ring from beta carotene, the other product is provitamin A. Accordingly, use of a plant-derived CCD provides a yield advantage.

Moreover, the use of plant-derived genes for production of compounds for use in the food industry is preferred.

In another aspect the present invention relates to transgenic organisms modified to express CCD polypeptides and therefore being capable of producing carotenoid cleavage compounds including β ionone, α ionone, pseudo ionone, theaspirone, dihydroactinidiolide, β damascenone, β damascone and β cyclocitral. The present invention further provides means and methods for the biotechnological production of carotenoid cleavage compounds which can be used as aroma additives for flavouring or perfuming.

In another aspect, the present invention relates to an isolated and/or purified novel CCD polypeptide or functional fragment thereof wherein said CCD polypeptide is derivable from the plant species Rubus idaeus (the raspberry plant). The invention also provides the nucleic acid sequence encoding said Rubus idaeus CCD polypeptide

It is to be noted that the present invention provides a new and useful use of enzymes derived from Arabidopsis thaliana, Rubus idaeus, Zea mais and tomato. The present invention further comprises methods for making specific ionones that hitherto has not been disclosed or suggested in the art.

Aspects of the present invention are presented in the claims and in the following commentary.

For ease of reference, these and further aspects of the present invention are now discussed under appropriate section headings. However, the teachings under each section are not necessarily limited to each particular section.

As used with reference to the present invention, the terms “produce”, “producing”, “produced”, “producable”, “production” are synonymous with the respective terms “prepare”, “preparing”, “prepared”, “preparation”, “generated”, “generation” and “preparable”.

As used with reference to the present invention, the terms “expression”, “expresses”, “expressed” and “expressable” are synonymous with the respective terms “transcription”, “transcribes”, “transcribed” and “transcribable”.

As used with reference to the present invention, the terms “transformation” and “transfection” refer to a method of introducing nucleic acid sequences into hosts, host cells, tissues or organs.

Other aspects concerning the nucleotide sequences which can be used in the present invention include: a construct comprising the sequences of the present invention; a vector comprising the sequences for use in the present invention; a plasmid comprising the sequences for use in the present invention; a transformed cell comprising the sequences for use in the present invention; a transformed tissue comprising the sequences for use in the present invention; a transformed organ comprising the sequences for use in the present invention; a transformed host comprising the sequences for use in the present invention; a transformed organism comprising the sequences for use in the present invention. The present invention also encompasses methods of expressing the nucleotide sequence for use in the present invention using the same, such as expression in a host cell; including methods for transferring same. The present invention flirter encompasses methods of isolating the nucleotide sequence, such as isolating from a host cell.

Other aspects concerning the amino acid sequences for use in the present invention include: a construct encoding the amino acid sequences for use in the present invention; a vector encoding the amino acid sequences for use in the present invention; a plasmid encoding the amino acid sequences for use in the present invention; a transformed cell expressing the amino acid sequences for use in the present invention; a transformed tissue expressing the amino acid sequences for use in the present invention; a transformed organ expressing the amino acid sequences for use in the present invention; a transformed host expressing the amino acid sequences for use in the present invention; a transformed organism expressing the amino acid sequences for use in the present invention. The present invention also encompasses methods of purifying the amino acid sequence for use in the present invention using the same, such as expression in a host cell; including methods of transferring same, and then purifying said sequence.

### BRIEF DESCRIPTION OF THE DRAWINGS

FIG. 1 shows a scheme for carotenoid and apocarotenoid biosynthesis.

FIG. 2A shows the GC-MS trace from the residue extracted from BL21-pAC-BETA-pRSETA.

FIG. 2B shows the GC-MS trace from the residue extracted from BL21-pAC-BETA-pRSETA-AtCCD. The arrow 1 indicates β ionone.

FIG. 3 shows the GC-MS trace from pRSETA-RiCCD#1. Arrow 1 indicates β ionone while arrow 2 indicates pseudo ionone.

FIG. 4 shows a sequence alignment between SEQ ID NO: 8 Y14387 (retrieved from the database), and SEQ ID NO: 9 which is the sequence determined and cloned as described herein.

### DETAILED DISCLOSURE OF INVENTION

In one aspect, the invention provides a plant-derived enzyme involved in carotenoid biosynthesis.

In particular, the invention provides a plasmid or vector system comprising a plant-derived CCD as described herein or a homologue or derivative thereof. Preferably, the plasmid or vector system comprises a nucleic acid sequence as set out in any of SEQ ID Nos: 1, 3 or 5 or a sequence that is at least 75% homologous thereto or an effective fragment thereof. Suitably the plasmid or vector system is an expression vector for the expression of any of the enzymes encoded by a nucleic acid sequence as set out in any of SEQ ID Nos: 1, 3 or 5 or a sequence that is at least 75%, 80%, 85%, 90%, 95% or 99% homologous (identical) thereto in a microorganism. Suitable expression vectors are described herein.

In one embodiment, the plant-derived enzyme is an epsilon cyclase gene, suitably derived from tomato. Preferably, the epsilon cyclase gene has a sequence as set out in SEQ ID NO: 9.

In a further aspect of the invention there is provided a host cell transformed or transfected with a nucleic acid encoding a plant-derived enzyme and, preferably a plant-derived CCD enzyme. Preferably, the plant-derived CCD is a CCD as described herein or a homologue or derivative thereof. Suitably, said plant-derived CCD enzyme comprises an amino acid sequence, or functional fragment thereof, as set out in any of SEQ ID Nos: 2, 4 or 6 or a sequence that is at least 75% homologous (identical) thereto. Preferably, said host cell produces a carotenoid cleavage compound.

In one embodiment, the nucleotide sequence which can be used in the present invention is obtainable from (though it does not have to be actually obtained from) Arabidopsis thaliana strain W, Rubus idaeus strain Tulameen or Zea mais cultivar Dent MBS847, although it will be recognised that enzymes isolated and/or purified from equivalent strains may equally be used.

In another embodiment the nucleotide sequence which can be used in a host cell of the present invention is obtainable from tomato. Suitably the nucleotide sequence is as set out in SEQ ID NO: 10.

Suitably the host cell is derived from animal or plant cells, seeds, tissues, and whole plants. In a preferred embodiment, the host cell is a microorganism including bacteria and fungi, including yeast. In a particularly preferred embodiment the host cell is a prokaryotic bacterial cell. Suitable bacterial host cells include bacteria from different procaryotic taxonomic groups including proteobacteria, including members of the alpha, beta, gamma, delta and epsilon subdivision, gram-positive bacteria such as Actinomycetes, Firmicutes, Clostridium and relatives, flavobacteria, cyanobacteria, green sulfur bacteria, green non-sulfur bacteria, and archaea. Particularly preferred are the Enterobacteriaceae such as Escherichia coli proteobacteria belonging to the gamma subdivision. Other suitable bacteria include Brevibacterium linens and Brevibacterium erythrogenes.

Suitable fungal host cells include yeast selected from the group consisting of Ascomycota including Saccharomycetes such as Pichia and Saccharomyces, and anamorphic Ascomycota including Aspergillus. Other suitable fungi include Phycomyces blakesleeanus and carotenogenic yeast strains such as Phaffia rhodozyma (Xanthophyllomyces dendrorhous).

Other suitable eucaryotic host cells include insect cells such as SF9, SF21, Trychplusiani and M121 cells. For example, the polypeptides according to the invention can advantageously be expressed in insect cell systems. As well as expression in insect cells in culture, CCD genes can be expressed in whole insect organisms. Virus vectors such as baculovirus allow infection of entire insects. Large insects, such as silk moths, provide a high yield of heterologous protein. The protein can be extracted from the insects according to conventional extraction techniques. Expression vectors suitable for use in the invention include all vectors which are capable of expressing foreign proteins in insect cell lines.

Other host cells include plant cells selected from the group consisting of protoplasts, cells, calli, tissues, organs, seeds, embryos, ovules, zygotes, etc. The invention also provides whole plants that have been transformed and comprise the recombinant DNA of the invention.

The term “plant” generally includes eukaryotic alga, embryophytes including Bryophyta, Pteridophyta and Spermatophyta such as Gymnospermae and Angiospermae. Generally, the present invention is applicable in species cultivated for food, drugs, beverages, and the like.

In one embodiment, the host cell expresses one of the precursor components of the carotenoid synthetic pathway such that, on introduction of the CCD, cleavage of that precursor to generate the desired carotenoid cleavage compound will occur. Such precursors include, for example, β carotene, lycopene, delta carotene and so forth.

Many microbes are known to accumulate carotenoids in large amounts. Many photosynthetic and non-photosynthetic bacteria and fungi are known for this property.

Suitable bacteria include, for example, Erwinia spp., Gordonia spp., Rubrivivax spp., Rhodobacter spp., Eiythrobacter spp., Rhodotorula spp., Deinococcus radiodurans, Brevibacterium linens, Sphingomonas spp. and Xanthomonas spp., Suitable fungi include, for example, Phycomyces sp. such as Phycomyces blakesleeanus, Neurospera crassa and Phaffia rhodozyma (Xanthophyllomyces dendrorhous).

When one of the CCD enzymes is expressed in such micro-organisms, compounds like β ionone, a ionone and pseudo ionone can be produced in large quantities. Accordingly, in one embodiment the host cell is one that normally accumulates a carotenoid. In a particularly preferred embodiment, the host cell is one that normally accumulates geranylgeranyl diphosphate (GGDP).

Alternatively, other bacteria can be provided with carotenoid-biosynthesis genes from plant or microbial source, and can, in combination with a CCD gene, lead to production of β ionone, α ionone or pseudo ionone, or other carotenoid-derived flavor compounds.

Thus, in one embodiment, there is provided a host cell in accordance with the invention further comprising a transgene encoding a carotenoid biosynthesis enzyme. Suitable enzymes include enzymes that convert geranylgeranyl diphosphate to β carotene, crtB and crtL-e.

In one embodiment the crtL-e is derived from tomato and, suitably, has a sequence set out in SEQ ID NO:9.

In a further aspect of the invention there is provided a method of producing a carotenoid cleavage compound comprising treating a carotenoid with a plant-derived carotenoid cleavage dioxygenase (CCD). Preferably, the method is characterised in that the enzyme comprises at least any one of the amino acid sequences shown as SEQ ID NOs: 2, 4 or 6 or a sequence having at least 75% identity (homology) thereto or an effective fragment thereof. Suitably, the carotenoid is α, β, γ or δ carotene or lycopene and the carotenoid cleavage compound is α or β ionone, pseudo ionone, safranal, theaspirone or damascenone. In a preferred embodiment, the method is an in vivo biotechnological process.

In a yet further aspect of the invention, there is provided a method for producing a carotenoid cleavage compound which comprises:
a) providing a host cell that produces a carotenoid wherein the host cell comprises expressible transgenes comprising a plant-derived CCD;
b) culturing the transgenic organism under conditions suitable for expression of the transgene; and
c) recovering the carotenoid cleavage compound from the culture.

In one embodiment, the host cell further comprises an expressible transgene comprising another carotenoid biosynthetic enzyme. Suitable enzymes include epsilon cyclase. In one embodiment, the epsilon cyclase gene is derived from tomato. Suitably the gene has a sequence as set out in SEQ ID NO:9.

Suitably, the host cell is a microorganism. In a preferred embodiment of any aspect of the invention, the carotenoid cleavage compound is β ionone. More preferably, the host cell is a microorganism that normally accumulates or produces a carotenoid selected from β carotene or γ carotene or cc carotene.

In another embodiment of any aspect of the invention, the plant-derived CCD is selected from Arabidopsis thaliana CCD (AtCCD), Rubus idaeus CCD (RiCCD) or Zea mais CCD (ZmCCD) enzyme (or any other enzyme that is more than 60% identical).

In an alternative embodiment of any aspect of the invention, the host cell is a microorganism that normally accumulates or produces lycopene. Suitably, in this embodiment, the expressible transgene is RiCCD and the carotenoid cleavage compound is pseudo ionone.

In a further embodiment of any aspect of the invention, the host cell is a microorganism that normally accumulates δ carotene or α carotene. Suitably, in this embodiment, the expressible transgene is AtCCD, RiCCD or ZmCCD and the carotenoid cleavage compound is a ionone. In this embodiment, the host cell may suitably also express epsilon cyclase.

In another embodiment, the host cell is derived from a transgenic plant, for instance tomato. In this embodiment the AtCCD, RiCCD or ZmCCD enzyme is expressed in organs (e.g. fruits) and organelles (e.g. chromoplasts) that accumulate carotenoids and α ionone, β ionone and pseudo ionone is produced.

The present invention features an enzyme comprising the amino acid sequence corresponding to Rubus idaeus CCD or a functional equivalent thereof or an effective fragment thereof.

The term “corresponding to Rubus idaeus CCD” means that the enzyme need not have been obtained from a source of Rubus idaeus. Instead, the enzyme has to have the same sequence as that of Rubus idaeus CCD.

The term “functional equivalent thereof” means that the enzyme has to have the same functional characteristics as that of Rubus idaeus CCD.

Preferably the enzyme of this aspect of the present invention has the same sequence or a sequence that is at least 75% identical (homologous) to that of Rubus idaeus CCD.

Suitably, the enzyme comprises the amino acid sequence as shown in SEQ ID NO: 4 or a sequence having at least 75% identity (homology) thereto or an effective fragment thereof. In a preferred embodiment, the invention provides an isolated and/or purified polypeptide having the amino acid sequence as set out in SEQ ID NO: 4 or a sequence having at least 75% identity (homology) thereto or an effective fragment thereof.

In another aspect, the invention provides an isolated and/or purified nucleic acid molecule or nucleotide sequence coding for the enzyme of Rubus idaeus CCD, or a homologue thereof. Suitably said isolated and/or purified nucleic acid molecule encodes a polypeptide comprising the amino acid sequence as shown in SEQ ID NO: 4 or a sequence having at least 75% identity (homology) thereto or an effective fragment thereof. In another embodiment, the invention provides an isolated and/or purified nucleic acid molecule comprising a nucleotide sequence that is the same as, or is complementary to, or contains any suitable codon substitutions for any of those of SEQ ID NO: 3 or comprises a sequence which has at least 75%, 80%, 85%, 90%, 95% or 99% sequence homology with SEQ ID NO: 3.

In a yet further aspect, the invention relates to a nucleotide sequence and to the use of a nucleotide sequence shown as:

(a) the nucleotide sequence presented as SEQ ID No.3,

(b) a nucleotide sequence that is a variant, homologue, derivative or fragment of the nucleotide sequence presented as SEQ ID No. 3;

(c) a nucleotide sequence that is the complement of the nucleotide sequence set out in SEQ ID No. 3;

(d) a nucleotide sequence that is the complement of a variant, homologue, derivative or fragment of the nucleotide sequence presented as SEQ ID No 3;

(e) a nucleotide sequence that is capable of hybridising to the nucleotide sequence set out in SEQ ID No. 3;

(f) a nucleotide sequence that is capable of hybridising to a variant, homologue, derivative or fragment of the nucleotide sequence presented as SEQ ID No. 3;

(g) a nucleotide sequence that is the complement of a nucleotide sequence that is capable of hybridising to the nucleotide sequence set out in SEQ ID No. 3;

(h) a nucleotide sequence that is the complement of a nucleotide sequence that is capable of hybridising to a variant, homologue, derivative or fragment of the nucleotide sequence presented as SEQ ID No. 3;

(i) a nucleotide sequence that is capable of hybridising to the complement of the nucleotide sequence set out in SEQ ID No.3;

(j) a nucleotide sequence that is capable of hybridising to the complement of a variant, homologue, derivative or fragment of the nucleotide sequence presented as SEQ ID No. 3.

The nucleotide sequence of the present invention may comprise sequences that encode for SEQ ID No. 3 or a variant, homologue or derivative thereof.

In another aspect of the invention there is provided a CrtL-e enzyme comprising the amino acid sequence as shown in SEQ ID NO: 10 or an effective fragment thereof.

In a further aspect there is provided an isolated nucleic acid molecule coding for the enzyme CrtL-e enzyme having the sequence set out in SEQ ID NO: 10. Suitably, said isolated nucleic acid molecule comprises a nucleotide sequence that is the same as, or is complementary to, or contains any suitable codon substitutions for any of those of SEQ ID NO: 9.

Preferable Aspects

Preferable aspects are presented in the accompanying claims and in the following description and Examples section

Additional Advantages

The present invention is advantageous as it provides a microbiological process for the synthesis of flavouring and perfume compounds.

The products of the present invention may be used in various applications in the food industry—such as in bakery and drink products, they may also be used in other applications such as a pharmaceutical composition, or even in the chemical industry.

Carotenoids

Carotenoids form a large group of structurally diverse higher terpene pigments that are widespread in plants and microorganisms. They function in species-specific coloration, photoprotection and light harvesting. Very potent aroma compounds are derived from carotenoids, attracting the attention of chemists and flavorists. A wide variety of carotenoid aroma compounds have been isolated from plant extracts.

Examples of carotenoid aroma compounds or aroma precursor compounds include saffron, which is obtained from the flowers of Crocus sativus, β ionone, α ionone, pseudo ionone, theaspirone, dihydroactinidiolide, damascenone, damascone and β cyclocitral. These degradation products serve in plants as pollinator attractants, anti-fungals or to deter herbivores. The 13 carbon ionones are found in many fruit flavours (raspberry, blackberry, blackcurrant, peach, apricot, melon, tomato, quince, starfruit), plant odours (violet, black tea, tobacco, carrot, vanilla, rose, green tea, osmanthus) and mushrooms.

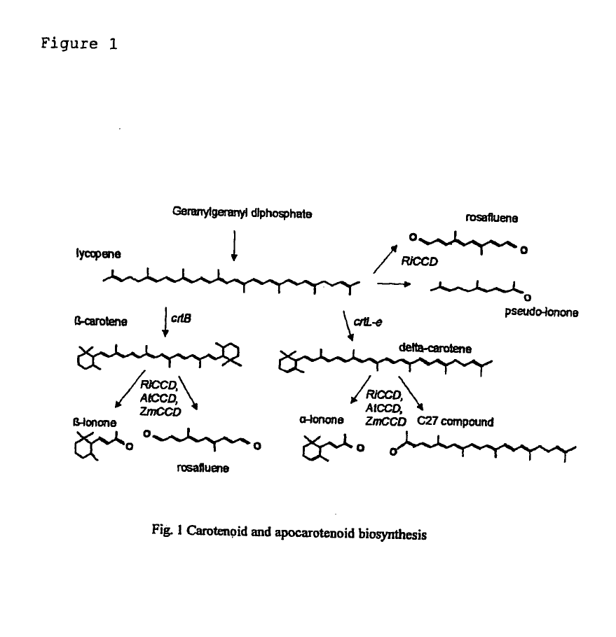

Carotenoid biosynthetic genes express enzymes that catalyse the biosynthetic pathways in vivo. The biosynthesis of carotenoids derives from the synthesis of geranylgeranyl diphosphate, two units of which are condensed to form phytoene. By removal of double bonds in phytoene, it is converted into lycopene. Lycopene can be processed into a number of derivatives, among which are β carotene (made from lycopene by action of the enzyme lycopene β cyclase; crtY) and delta carotene (made from lycopene by action of crtL-e; lycopene epsilon cyclase). If lycopene is cleaved at the 9-10 double bond and at the 9′-10′ double bond, the predicted products are rosafluene and pseudo ionone. If β carotene is cleaved at the 9-10 double bond and at the 9′-10′ double bond, the predicted products are rosafluene and β ionone. If delta carotene is cleaved at the 9-10 double bond, the predicted products are alfa ionone and a C27 compound. This biosynthetic pathway is summarised in FIG. 1.

Carotenoid-cleavage-dioxygenase (CCD) genes encode the enzymes that cleave carotenoids such as β carotene, delta carotene and lycopene. A number of CCD genes from different sources including certain plants and animals have been described.

The Arabidopsis thaliana (At) CCD enzyme has been described to cleave a number of carotenoids in vitro, to produce rosafluene from β carotene and other carotenoids in vitro. Similar observations were made for the homologous enzyme from pea (PvCCD1). However, formation of β ionone was not reported (Schwartz et al. (2001) J Biol Chem 276(27):25208-11). Similarly, a CCD gene from Crocus sativus (CsCCD) has been shown to cleave zeaxanthin in vitro to form rosafluene. Again, however, no formation of β ionone was reported (Bouvier et al. (2003a) Plant Cell. 15(1):47-62).

Other reports describe CCD genes from maize (VP14; Schwartz et al. (1997) Science. 276(5320):1872-4.), mouse (βCD; Redmond et al., (2001) J Biol Chem. 276(9):6560-5), Crocus sativus (CsZCD; Bouvier et al. (2003a) Plant Cell. 15(1):47-62), Bixa orellana (BoLCD; Bouvier et al. (2003b) Science. 300(5628):2089-91) and Pseudomonas paucimobilis (Kamoda & Saburi (1993) Biosci Biotechnol Biochem. 57(6):926-30.). However, due to the position of cleavage within the carotenoids molecule taken by these enzymes, they are therefore not useful for the production of certain compounds such as β ionone, a ionone or pseudo ionone.

A distantly related CCD gene (less than 30% identical) from mouse has been described, which, upon expression in a β-carotene producing bacterium, was shown to produce β ionone (Kiefer et al., (2001) J. Biol. Chem. 276, 14110-6). Other CCD genes (either from plant origin or from other organisms) have not previously been reported to produce β-, α- or pseudo ionone in microorganisms.

Isolated

In one aspect, preferably the sequence is in an isolated form. The term “isolated” means that the sequence is at least substantially free from at least one other component with which the sequence is naturally associated in nature and as found in nature.

Purified

In one aspect, preferably the sequence is in a purified form. The term “purified” means that the sequence is in a relatively pure state—e.g. at least about 90% pure, or at least about 95% pure or at least about 98% pure.

Nucleotide Sequence

The scope of the present invention encompasses nucleotide sequences encoding enzymes having the specific properties as defined herein.

The term “nucleotide sequence” as used herein refers to an oligonucleotide sequence, nucleic acid or polynucleotide sequence, and variant, homologues, fragments and derivatives thereof (such as portions thereof). The nucleotide sequence may be of genomic or synthetic or recombinant origin, which may be double-stranded or single-stranded whether representing the sense or anti-sense strand.

The term “nucleotide sequence” or “nucleic acid molecule” in relation to the present invention includes genomic DNA, cDNA, synthetic DNA, and RNA. Preferably it means DNA, more preferably cDNA sequence coding for the present invention.

In a preferred embodiment, the nucleotide sequence when relating to and when encompassed by the per se scope of the present invention does not include the native nucleotide sequence according to the present invention when in its natural environment and when it is linked to its naturally associated sequence(s) that is/are also in its/their natural environment. For ease of reference, we shall call this preferred embodiment the “non-native nucleotide sequence”. In this regard, the term “native nucleotide sequence” means an entire nucleotide sequence that is in its native environment and when operatively linked to an entire promoter with which it is naturally associated, which promoter is also in its native environment. However, the amino acid sequence encompassed by scope the present invention can be isolated and/or purified post expression of a nucleotide sequence in its native organism. Preferably, however, the amino acid sequence encompassed by scope of the present invention may be expressed by a nucleotide sequence in its native organism but wherein the nucleotide sequence is not under the control of the promoter with which it is aurally associated within that organism.

Preparation of a Nucleotide Sequence

Typically, the nucleotide sequence encompassed by scope of the present invention or the nucleotide sequences for use in the present invention are prepared using recombinant DNA techniques (i.e. recombinant DNA). However, in an alternative embodiment of the invention, the nucleotide sequence could be synthesised, in whole or in part, using chemical methods well known in the art (see Caruthers M H et al., (1980) Nuc Acids Res Symp Ser 215-23 and Horn T et al., (1980) Nuc Acids Res Symp Ser 225-232).

A nucleotide sequence encoding either an enzyme which has the specific properties as defined herein or an enzyme which is suitable for modification may be identified and/or isolated and/or purified from any cell or organism producing said enzyme. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the organism producing the enzyme. If the amino acid sequence of the enzyme or a part of the amino acid sequence of the enzyme is known, labelled oligonucleotide probes may be synthesised and used to identify enzyme-encoding clones from the genomic library prepared from the organism. Alternatively, a labelled oligonucleotide probe containing sequences homologous to another known enzyme gene could be used to identify enzyme-encoding clones. In the latter case, hybridisation and washing conditions of lower stringency are used.

Alternatively, enzyme-encoding clones could be identified by inserting fragments of genomic DNA into an expression vector, such as a plasmid, transforming enzyme-negative bacteria with the resulting genomic DNA library, and then plating the transformed bacteria onto agar plates containing a substrate for the enzyme (e.g. maltose for a glucosidase (maltase) producing enzyme), thereby allowing clones expressing the enzyme to be identified.

In a yet further alternative, the nucleotide sequence encoding the enzyme may be prepared synthetically by established standard methods, e.g. the phosphoroamidite method described by Beucage S. L. et al., (1981) Tetrahedron Letters 22, p 1859-1869, or the method described by Matthes et al, (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al, (Science (1988) 239, pp 487-491).

Due to degeneracy in the genetic code, nucleotide sequences may be readily produced in which the triplet codon usage, for some or all of the amino acids encoded by the original nucleotide sequence, has been changed thereby producing a nucleotide sequence with low homology to the original nucleotide sequence but which encodes the same, or a variant, amino acid sequence as encoded by the original nucleotide sequence. For example, for most amino acids the degeneracy of the genetic code is at the third position in the triplet codon (wobble position) (for reference see Stryer, Lubert, Biochemistry, Third Edition, Freeman Press, ISBN 0-7167-1920-7) therefore, a nucleotide sequence in which all triplet codons have been “wobbled” in the third position would be about 66% identical to the original nucleotide sequence however, the amended nucleotide sequence would encode for the same, or a variant, primary amino acid sequence as the original nucleotide sequence.

Therefore, the present invention further relates to any nucleotide sequence that has alternative triplet codon usage for at least one amino acid encoding triplet codon, but which encodes the same, or a variant, polypeptide sequence as the polypeptide sequence encoded by the original nucleotide sequence.

Furthermore, specific organisms typically have a bias as to which triplet codons are used to encode amino acids. Preferred codon usage tables are widely available, and can be used to prepare codon optimised genes. Such codon optimisation techniques are routinely used to optimise expression of transgenes in a heterologous host.

Molecular Evolution

Once an enzyme-encoding nucleotide sequence has been isolated and/or purified, or a putative enzyme-encoding nucleotide sequence has been identified, it may be desirable to modify the selected nucleotide sequence, for example it may be desirable to mutate the sequence in order to prepare an enzyme in accordance with the present invention.

Mutations may be introduced using synthetic oligonucleotides. These oligonucleotides contain nucleotide sequences flanking the desired mutation sites.

A suitable method is disclosed in Morinaga et al (Biotechnology (1984) 2, p 646-649). Another method of introducing mutations into enzyme-encoding nucleotide sequences is described in Nelson and Long (Analytical Biochemistry (1989), 180, p 147-151).

Instead of site directed mutagenesis, such as described above, one can introduce mutations randomly for instance using a commercial kit such as the GeneMorph PCR mutagenesis kit from Stratagene, or the Diversify PCR random mutagenesis kit from Clontech.

A third method to obtain novel sequences is to fragment non-identical nucleotide sequences, either by using any number of restriction enzymes or an enzyme such as Dnase I, and reassembling full nucleotide sequences coding for functional proteins. Alternatively one can use one or multiple non-identical nucleotide sequences and introduce mutations during the reassembly of the full nucleotide sequence.

Thus, it is possible to produce numerous site directed or random mutations into a nucleotide sequence, either in vivo or in vitro, and to subsequently screen for improved functionality of the encoded polypeptide by various means.

As a non-limiting example, mutations or natural variants of a polynucleotide sequence can be recombined with either the wildtype or other mutations or natural variants to produce new variants. Such new variants can also be screened for improved functionality of the encoded polypeptide. The production of new preferred variants may be achieved by various methods well established in the art, for example the Error Threshold Mutagenesis (WO 92/18645), oligonucleotide mediated random mutagenesis (U.S. Pat. No. 5,723,323), DNA shuffling (U.S. Pat. No. 5,605,793), exo-mediated gene assembly (WO/58517).

The application of the above-mentioned and similar molecular evolution methods allows the identification and selection of variants of the enzymes of the present invention which have preferred characteristics without any prior knowledge of protein structure or function, and allows the production of non-predictable but beneficial mutations or variants. There are numerous examples of the application of molecular evolution in the art for the optimisation or alteration of enzyme activity, such examples include, but are not limited to one or more of the following: optimised expression and/or activity in a host cell or in vitro, increased enzymatic activity, altered substrate and/or product specificity, increased or decreased enzymatic or structural stability, altered enzymatic activity/specificity in preferred environmental conditions, e.g. temperature, pH, substrate

Amino Acid Sequences

The scope of the present invention also encompasses amino acid sequences of enzymes having the specific properties as defined herein.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The amino acid sequence may be prepared/isolated from a suitable source, or it may be made synthetically or it may be prepared by use of recombinant DNA techniques.

The enzyme encompassed in the present invention may be used in conjunction with other enzymes. Thus the present invention also covers a combination of enzymes wherein the combination comprises the enzyme of the present invention and another enzyme, which may be another enzyme according to the present invention. This aspect is discussed in a later section.

Preferably the amino acid sequence when relating to and when encompassed by the per se scope of the present invention is not a native enzyme. In this regard, the term “native enzyme” means an entire enzyme that is in its native environment and when it has been expressed by its native nucleotide sequence.

Variants/Homologues/Derivatives

The present invention also encompasses the use of variants, homologues and derivatives of any amino acid sequence of an enzyme or of any nucleotide sequence encoding such an enzyme.

Here, the term “homologue” means an entity having a certain homology with the amino acid sequences and the nucleotide sequences. Here, the term “homology” can be equated with “identity”.

In the present context, a homologous amino acid sequence is taken to include an amino acid sequence which may be at least 75, 80, 81, 85 or 90% identical, preferably at least 95, 96, 97, 98 or 99% identical to the sequence. Typically, the homologues will comprise the same active sites etc.—e.g. as the subject amino acid sequence. Although homology can also be considered in terms of similarity (i.e. amino acid residues having similar chemical properties/functions), in the context of the present invention it is preferred to express homology in terms of sequence identity.

By “functional fragment” is meant a fragment of the polypeptide that retains that characteristic properties of that polypeptide. In the context of the present invention, a functional fragment of a CCD enzyme is a fragment that retains the carotenoid cleavage capability of the whole protein.

In the present context, an homologous nucleotide sequence is taken to include a nucleotide sequence which may be at least 75, 80, 81, 85 or 90% identical, preferably at least 95, 96, 97, 98 or 99% identical to a nucleotide sequence encoding an enzyme of the present invention (the subject sequence). Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

For the amino acid sequences and the nucleotide sequences, homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an other identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and 4 for each extension.

Calculation of maximum % homology therefore firstly requires the production of an optimal alignment, taking into consideration gap penalties. A suitable computer program for carrying out such an alignment is the GCG Wisconsin Bestfit package (Devereux et al 1984 Nuc. Acids Research 12 p 387). Examples of other software than can perform sequence comparisons include, but are not limited to, the BLAST package (see Ausubel et al., 1999 Short Protocols in Molecular Biology, 4^{th} Ed—Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60).

However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins D G & Sharp P M (1988), Gene 73(1), 237-244).

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids can be grouped together based on the properties of their side chain alone. However it is more useful to include mutation data as well. The sets of amino acids thus derived are likely to be conserved for structural reasons. These sets can be described in the form of a Venn diagram (Livingstone C. D. and Barton G. J. (1993) “Protein sequence alignments: a strategy for the hierarchical analysis of residue conservation” Comput. Appl Biosci. 9: 745-756) (Taylor W Y (1986) “The classification of amino acid conservation” J. Theor. Biol. 119; 205-218). Conservative substitutions may be made, for example according to the table below which describes a generally accepted Venn diagram grouping of amino acids.

| SET | | SUB-SET | |
|---|---|---|---|
| Hydrophobic | F W Y H K M I L V A G C | Aromatic | F W Y H |
| | | Aliphatic | I L V |
| Polar | W Y H K R E D C S T N Q | Charged | H K R E D |
| | | Positively | H K R |
| | | charged | |
| | | Negatively | E D |
| | | charged | |
| Small | V C A G S P T N D | Tiny | A G S |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and stain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. The primers may be designed to contain suitable restriction enzyme recognition sites so that the amplified DNA can be cloned into a suitable cloning vector.

Biologically Active

Preferably, the variant sequences etc. are at least as biologically active as the sequences presented herein.

As used herein “biologically active” refers to a sequence having a similar structural function (but not necessarily to the same degree), and/or similar regulatory function (but not necessarily to the same degree), and/or similar biochemical function (but not necessarily to the same degree) of the naturally occurring sequence.

Hybridisation

The present invention also encompasses sequences that are complementary to the nucleic acid sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under stringent conditions (e.g. 50° C. and 0.2×SSC {1×SSC=0.15 M NaCl, 0.015 M Na₃citrate pH 7.0}) to the nucleotide sequences presented herein.

More preferably, the term “variant” encompasses sequences that are complementary to sequences that are capable of hybridising under high stringent conditions (e.g. 65° C. and 0.1×SSC {1×SSC=0.15 M NaCl, 0.015 M Na₃citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

Also included within the scope of the present invention are polynucleotide sequences that are capable of hybridising to the nucleotide sequences presented herein under conditions of intermediate to maximal stringency.

In a preferred aspect, the present invention covers nucleotide sequences that can hybridise to the nucleotide sequence of the present invention, or the complement thereof, under stringent conditions (e.g. 50° C. and 0.2×SSC).

In a more preferred aspect, the present invention covers nucleotide sequences that can hybridise to the nucleotide sequence of the present invention, or the complement thereof, under high stringent conditions (e.g. 65° C. and 0.1×SSC).

Site-Directed Mutagenesis

Once au enzyme-encoding nucleotide sequence has been isolated and/or purified, or a putative enzyme-encoding nucleotide sequence has been identified, it may be desirable to mutate the sequence in order to prepare an enzyme of the present invention.

Mutations may be introduced using synthetic oligonucleotides. These oligonucleotides contain nucleotide sequences flanking the desired mutation sites.

A suitable method is disclosed in Morinaga et al., (Biotechnology (1984) 2, p 646-649). Another method of introducing mutations into enzyme-encoding nucleotide sequences is described in Nelson and Long (Analytical Biochemistry (1989), 180, p 147-151). A further method is described in Sarkar and Sommer (Biotechniques (1990), 8, p 404-407—“The megaprimer method of site directed mutagenesis”).

Recombinant

In one aspect the sequence for use in the present invention is a recombinant sequence—i.e. a sequence that has been prepared using recombinant DNA techniques.

These recombinant DNA techniques are within the capabilities of a person of ordinary skill in the art. Such techniques are explained in the literature, for example, J. Sambrook, E. F. Fritsch, and T. Maniatis, 1989, Molecular Cloning: A Laboratory Manual, Second Edition, Books 1-3, Cold Spring Harbor Laboratory Press.

Synthetic

In one aspect the sequence for use in the present invention is a synthetic sequence—i.e. a sequence that has been prepared by in vitro chemical or enzymatic synthesis. It includes, but is not limited to, sequences made with optimal codon usage for host organisms—such as the methylotrophic yeasts Pichia and Hansenula.

Expression of Enzymes

The nucleotide sequence for use in the present invention may be incorporated into a recombinant replicable vector. The vector may be used to replicate and express the nucleotide sequence, in enzyme form, in and/or from a compatible host cell.

Expression may be controlled using control sequences e.g. regulatory sequences.

The enzyme produced by a host recombinant cell by expression of the nucleotide sequence may be secreted or may be contained intracellularly depending on the sequence and/or the vector used. The coding sequences may be designed with signal sequences which direct secretion of the substance coding sequences through a particular prokaryotic or eukaryotic cell membrane.

Expression Vector

The terms “plasmid”, “vector system” or “expression vector” means a construct capable of in vivo or in vitro expression. In the context of the present invention, these constructs may be used to introduce genes encoding enzymes into host cells. Suitably, the genes whose expression is introduced may be referred to as “expressible transgenes”.

Preferably, the expression vector is incorporated into the genome of a suitable host organism. The term “incorporated” preferably covers stable incorporation into the genome.

The nucleotide sequences described herein including the nucleotide sequence of the present invention may be present in a vector in which the nucleotide sequence is operably linked to regulatory sequences capable of providing for the expression of the nucleotide sequence by a suitable host organism.

The vectors for use in the present invention may be transformed into a suitable host cell as described below to provide for expression of a polypeptide of the present invention.

The choice of vector e.g. a plasmid, cosmid, or phage vector will often depend on the host cell into which it is to be introduced.

The vectors for use in the present invention may contain one or more selectable marker genes—such as a gene, which confers antibiotic resistance e.g. ampicillin, kanamycin, chloramphenicol or tetracyclin resistance. Alternatively, the selection may be accomplished by cotransformation (as described in WO91/17243).

Vectors may be used in vitro, for example for the production of RNA or used to transfect, transform, transduce or infect a host cell.

Thus, in a further embodiment, the invention provides a method of making nucleotide sequences of the present invention by introducing a nucleotide sequence of the present invention into a replicable vector, introducing the vector into a compatible host cell, and growing the host cell under conditions which bring about replication of the vector.

The vector may further comprise a nucleotide sequence enabling the vector to replicate in the host cell in question. Examples of such sequences are the origins of replication of plasmids pUC19, pACYC177, pUB110, pE194, pAMB1 and pIJ702.

Regulatory Sequences

In some applications, the nucleotide sequence for use in the present invention is operably linked to a regulatory sequence which is capable of providing for the expression of the nucleotide sequence, such as by the chosen host cell. By way of example, the present invention covers a vector comprising the nucleotide sequence of the present invention operably linked to such a regulatory sequence, i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

The term “regulatory sequences” includes promoters and enhancers and other expression regulation signals.

The term “promoter” is used in the normal sense of the art, e.g. an RNA polymerase binding site.

Enhanced expression of the nucleotide sequence encoding the enzyme of the present invention may also be achieved by the selection of heterologous regulatory regions, e.g. promoter, secretion leader and terminator regions.

Preferably, the nucleotide sequence according to the present invention is operably linked to at least a promoter.

Examples of suitable promoters for directing the transcription of the nucleotide sequence in a bacterial, fungal or yeast host are well known in the art.

Constructs

The term “construct”—which is synonymous with terms such as “conjugate”, “cassette” and “hybrid”—includes a nucleotide sequence for use according to the present invention directly or indirectly attached to a promoter.

An example of an indirect attachment is the provision of a suitable spacer group such as an intron sequence, such as the Sh1-intron or the ADH intron, intermediate the promoter and the nucleotide sequence of the present invention. The same is true for the term “fused” in relation to the present invention which includes direct or indirect attachment. In some cases, the terms do not cover the natural combination of the nucleotide sequence coding for the protein ordinarily associated with the wild type gene promoter and when they are both in their natural environment.

The construct may even contain or express a marker, which allows for the selection of the genetic construct.

For some applications, preferably the construct of the present invention comprises at least the nucleotide sequence of the present invention operably linked to a promoter.

Host Cells

The term “host cell”—in relation to the present invention includes any cell that comprises either the nucleotide sequence or an expression vector as described above and which is used in the recombinant production of an enzyme having the specific properties as defined herein or in the methods of the present invention.

Thus, a further embodiment of the present invention provides host cells transformed or transfected with a nucleotide sequence that expresses the enzymes described in the present invention. The cells will be chosen to be compatible with the said vector and may for example be prokaryotic (for example bacterial), fungal, yeast or plant cells. Preferably, the host cells are not human cells.

Examples of suitable bacterial host organisms are gram positive or gram negative bacterial species.

Depending on the nature of the nucleotide sequence encoding the enzyme of the present invention, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

The host cell may be a protease deficient or protease minus strain.

The genotype of the host cell may be modified to improve expression.

Examples of host cell modifications include protease deficiency, supplementation of rare tRNA's, and modification of the reductive potential in the cytoplasm to enhance disulphide bond formation.

For example, the host cell E. coli may overexpress rare tRNA's to improve expression of heterologous proteins as exemplified/described in Kane (Curr Opin Biotechnol (1995), 6, 494-500 “Effects of rare codon clusters on high-level expression of heterologous proteins in E. coli”). The host cell may be deficient in a number of reducing enzymes thus favouring formation of stable disulphide bonds as exemplified/described in Bessette (Proc Natl Acad Sci USA (1999), 96, 13703-13708 “Efficient folding of proteins with multiple disulphide bonds in the Escherichia coli cytoplasm”).

Organism

The term “organism” in relation to the present invention includes any organism that could comprise the nucleotide sequence coding for the enzymes as described in the present invention and/or products obtained therefrom, and/or wherein a promoter can allow expression of the nucleotide sequence according to the present invention when present in the organism.

Suitable organisms may include a prokaryote, fungus, yeast or a plant.

The term “transgenic organism” in relation to the present invention includes any organism that comprises the nucleotide sequence coding for the enzymes as described in the present invention and/or the products obtained therefrom, and/or wherein a promoter can allow expression of the nucleotide sequence according to the present invention within the organism. Preferably the nucleotide sequence is incorporated in the genome of the organism.

The team “transgenic organism” does not cover native nucleotide coding sequences in their natural environment when they are under the control of their native promoter which is also in its natural environment.

Therefore, the transgenic organism of the present invention includes an organism comprising any one of, or combinations of, the nucleotide sequence coding for the enzymes as described in the present invention, constructs according to the present invention, vectors according to the present invention, plasmids according to the preset invention, cells according to the present invention, tissues according to the present invention, or the products thereof.

For example the transgenic organism may also comprise the nucleotide sequence coding for the enzyme of the present invention under the control of a heterologous promoter.

Transformation of Host Cells/Organism

As indicated earlier, the host organism can be a prokaryotic or a eukaryotic organism. Examples of suitable prokaryotic hosts include E. coli and Bacillus subtilis.

Teachings on the transformation of prokaryotic hosts is well documented in the art, for example see Sambrook et al (Molecular Cloning: A Laboratory Manual, 2nd edition, 1989, Cold Spring Harbor Laboratory Press). Other suitable methods are set out in the Examples herein. If a prokaryotic host is used then the nucleotide sequence may need to be suitably modified before transformation—such as by removal of introns.

Filamentous fungi cells may be transformed using various methods known in the art—such as a process involving protoplast formation and transformation of the protoplasts followed by regeneration of the cell wall in a manner known. The use of Aspergillus as a host microorganism is described in EP 0 238 023.

Another host organism can be a plant. A review of the general techniques used for transforming plants may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

General teachings on the transformation of fungi, yeasts and plants are presented in following sections.

Transformed Fungus

A host organism may be a fungus—such as a filamentous fungus. Examples of suitable such hosts include any member belonging to the genera Thermomyces, Acremonium, Aspergillus, Penicillium, Mucor, Neurospora, Trichoderma and the like.

Teachings on transforming filamentous fungi are reviewed in U.S. Pat. No. 5,741,665 which states that standard techniques for transformation of filamentous fingi and culturing the fungi are well known in the art. An extensive review of techniques as applied to N. crassa is found, for example in Davis and de Serres, Methods Enzymol (1971) 17A: 79-143.

Further teachings on transforming filamentous fungi are reviewed in U.S. Pat. No. 5,674,707.

In one aspect, the host organism can be of the genus Aspergillus, such as Aspergillus niger.

A transgenic Aspergillus according to the present invention can also be prepared by following, for example, the teachings of Turner G. 1994 (Vectors for genetic manipulation. In: Martinelli S. D., Kinghorn J. R. (Editors) Aspergillus: 50 years on. Progress in industrial microbiology vol 29. Elsevier Amsterdam 1994. pp. 641-666).

Gene expression in filamentous fungi has been reviewed in Punt et al. (2002) Trends Biotechnol 2002 May; 20(5):2006, Archer & Peberdy Crit. Rev Biotechnol (1997) 17(4):273-306.

Transformed Yeast

In another embodiment, the transgenic organism can be a yeast.

A review of the principles of heterologous gene expression in yeast are provided in, for example, Methods Mol Biol (1995), 49:341-54, and Curr Opin Biotechnol (1997) October; 8(5):554-60

In this regard, yeast—such as the species Saccharomyces cerevisi or Pichia pastoris (see FEMS Microbiol Rev (2000 24(1):4566), may be used as a vehicle for heterologous gene expression.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

For the transformation of yeast, several transformation protocols have been developed. For example, a transgenic Saccharomyces according to the present invention can be prepared by following the teachings of Hinnen et al., (1978, Proceedings of the National Academy of Sciences of the USA 75, 1929); Beggs, J D (1978, Nature, London, 275, 1220 104); and Ito, H et al (1983, J Bacteriology 153, 163-168).

The transformed yeast cells may be selected using various selective markers—such as auxotrophic markers dominant antibiotic resistance markers.

Transformed Plants/Plant Cells

A host organism suitable for the present invention may be a plant. A review of the general techniques may be found in articles by Potrykus (Annu Rev Plant Physiol Plant Mol Biol [1991] 42:205-225) and Christou (Agro-Food-Industry Hi-Tech March/April 1994 17-27).

Culturing and Production

Host cells transformed with the nucleotide sequence of the present invention may be cultured under conditions conducive to the production of the encoded enzyme and which facilitate recovery of the enzyme from the cells and/or culture medium.

The medium used to cultivate the cells may be any conventional medium suitable for growing the host cell in questions and obtaining expression of the enzyme.

The protein produced by a recombinant cell may be displayed on the surface of the cell.

The enzyme may be secreted from the host cells and may conveniently be recovered from the culture medium using well-known procedures.

Secretion

It may be desirable for the enzyme to be secreted from the expression host into the culture medium from where the enzyme may be more easily recovered. According to the present invention, the secretion leader sequence may be selected on the basis of the desired expression host. Hybrid signal sequences may also be used with the context of the present invention.

Typical examples of heterologous secretion leader sequences are those originating from the fungal amyloglucosidase (AG) gene (glaA—both 18 and 24 amino acid versions e.g. from Aspergillus), the α-factor gene (yeasts e.g. Saccharomyces, Kluyveromyces and Hansenula) or the α-amylase gene (Bacillus).

By way of example, the secretion of heterologous proteins in E. coli is reviewed in Methods Enzymol (1990) 182:132-43.

Detection

A variety of protocols for detecting and measuring the expression of the amino acid sequence are known in the art. Examples include enzyme-linked immunosorbent assay (ELISA), radioimmunoassay (RIA) and fluorescent activated cell sorting (FACS).

A wide variety of labels and conjugation techniques are known by those skilled in the art and can be used in various nucleic and amino acid assays.

A number of companies such as Pharmacia Biotech (Piscataway, N.J.), Promega (Madison, Wis.), and US Biochemical Corp (Cleveland, Ohio) supply commercial kits and protocols for these procedures.

Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

Also, recombinant immunoglobulins may be produced as shown in U.S. Pat. No. 4,816,567.

Fusion Proteins

The amino acid sequence for use according to the present invention may be produced as a fusion protein, for example to aid in extraction and purification. Examples of fusion protein partners include glutathione-S-transferase (GST), 6×His, GAL4 (DNA binding and/or transcriptional activation domains) and (β-galactosidase). It may also be convenient to include a proteolytic cleavage site between the fusion protein partner and the protein sequence of interest to allow removal of fusion protein sequences.

Preferably, the fusion protein will not hinder the activity of the protein sequence.

Gene fusion expression systems in E. coli have been reviewed in Curr Opin Biotechnol (1995) 6(5):501-6.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognised by a commercially available antibody.

Additional Sequences

The sequences for use according to the present invention may also be used in conjunction with one or more additional proteins of interest (POIs) or nucleotide sequences of interest (NOIs).

Non-limiting examples of POs include: proteins or enzymes involved in carotenoid metabolism or combinations thereof. The NOI may even be an antisense sequence for any of those sequences.

The POI may even be a fusion protein, for example to aid in extraction and purification or to enhance in vivo carotenoid cleavage.

The POI may even be fused to a secretion sequence.

Other sequences can also facilitate secretion or increase the yield of secreted POI. Such sequences could code for chaperone proteins as for example the product of Asergillus niger cyp B gene described in UK patent application 9821198.0.

The NOI coding for POI may be engineered in order to alter their activity for a number of reasons, including but not limited to, alterations, which modify the processing and/or expression of the expression product thereof. By way of further example, the NOI may also be modified to optimise expression in a particular host cell. Other sequence changes may be desired in order to introduce restriction enzyme recognition sites.

The NOI coding for the POI may include within it synthetic or modified nucleotides—such as methylphosphonate and phosphorothioate backbones.

The NOI coding for the POI may be modified to increase intracellular stability and half-life. Possible modifications include, but are not limited to, the addition of flanking sequences of the 5′ and/or 3′ ends of the molecule or the use of phosphorothioate or 2′ O-methyl rather than phosphodiesterase linkages within the backbone of the molecule.

Antibodies

One aspect of the present invention relates to amino acids that are immunologically reactive with the amino acid of SEQ ID No. 1 or SEQ ID No. 3.

Antibodies may be produced by standard techniques, such as by immunisation with the substance of the invention or by using a phage display library.

For the purposes of this invention, the term “antibody”, unless specified to the contrary, includes but is not limited to, polyclonal, monoclonal, chimeric, single chain, Fab fragments, fragments produced by a Fab expression library, as well as mimetics thereof. Such fragments include figments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies. Neutralising antibodies, i.e., those which inhibit biological activity of the substance polypeptides, are especially preferred for diagnostics and therapeutics.

If polyclonal antibodies are desired, a selected mammal (e.g., mouse, rabbit, goat, horse, etc.) is immunised with the sequence of the present invention (or a sequence comprising an immunological epitope thereof). Depending on the host species, various adjuvants may be used to increase immunological response.

Serum from the immunised animal is collected and treated according to known procedures. If serum containing polyclonal antibodies to the sequence of the present invention (or a sequence comprising an immunological epitope thereof) contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against the sequence of the present invention (or a sequence comprising an immunological epitope thereof) can also be readily produced by one skilled in the art and include, but are not limited to, the hybridoma technique Koehler and Milstein (1975 Nature 256:495-497), the human B-cell hybridoma technique (Kosbor et al., (1983) Immunol Today 4:72; Cote et al., (1983) Proc Natl Acad Sci 80:2026-2030) and the EBV-hybridoma technique (Cole et al., (1985) Monoclonal Antibodies and Cancer Therapy, Alan Rickman Liss Inc, pp 77-96).

In addition, techniques developed for the production of “chimeric antibodies”, the splicing of mouse antibody genes to human antibody genes to obtain a molecule with appropriate antigen specificity and biological activity may be used (Morrison et al., (1984) Proc Natl Acad Sci 81:6851-6855; Neuberger et al., (1984) Nature 312:604-608; Takeda et al., (1985) Nature 314:452-454).

Alternatively, techniques described for the production of single chain antibodies (U.S. Pat. No. 4,946,779) can be adapted to produce the substance specific single chain antibodies.

Antibody fragments which contain specific binding sites for the substance may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W D et al., (1989) Science 256:1275-1281).

Large Scale Application

In one preferred embodiment of the present invention, the amino acid sequence encoding a plant-derived CCD or the methods of the present invention are used for large scale applications. In particular, the methods of the present invention may be used for the large scale production of carotenoid cleavage compounds for industrial use in flavouring or perfume applications.

Typical cartenoid cleavage compounds include β ionone, α ionone, pseudo ionone, theaspirone, dihydroactinidiolide, β damascenone, β damascenone and β cyclocitral.

Preferably the amino acid sequence or carotenoid cleavage compound is produced in a quantity of from 1 g per litre to about 2 g per litre of the total cell culture volume after cultivation of the host organism.

Preferably the amino acid sequence or carotenoid cleavage compound is produced in a quantity of from 100 mg per litre to about 900 mg per litre of the total cell culture volume after cultivation of the host organism.

Preferably the amino acid sequence or carotenoid cleavage compound is produced in a quantity of from 250 mg per lire to about 500 mg per litre of the total cell culture volume after cultivation of the host organism.

Use of Enzymes and Carotenoid Cleavage Compounds

As stated above, the present invention also relates to the production of carotenoid cleavage compounds as described herein.

In particular, the present invention also relates to the use of the amino acid sequences as disclosed herein in the production of carotenoid cleavage compounds.

Thus, the present invention further relates to the use of the nucleotide sequences encoding plant-derived CCDs in generating expression vectors or systems for the expression of the plant-derived CCD enzymes.

In addition, the present invention relates to the use of such expression vectors or systems in the generation of host cells which express plant-derived CCDs.

The invention further relates to the use of modified host cells in the generation of precursors of carotenoid cleavage compounds or in the generation of specific carotenoid cleavage compounds.

Suitable carotenoid cleavage compounds include β ionone, a ionone, pseudo ionone, theaspirone, dihydroactinidiolide, β damascenone, β damascone and β cyclocitral.

The compounds can be used for improved aroma, flavour, mildness, consistency, texture, body, mouth feel, firmness, viscosity, gel fracture, structure and/or organoleptic properties and nutrition of products, such as food products, for consumption containing said compounds. Furthermore, the compounds can also be used in combination with other components of products for consumption to deliver said improvements.

Accordingly, the invention further provides the use of an amino acid sequence encoding a plant-derived CCD or a host cell expressing a plant-derived CCD to produce a carotenoid cleavage compound for use in the manufacture of a food product. In one aspect, there is provided a use of an amino acid sequence as described herein in the manufacture of a food product. In another aspect, there is provided a use of a host cell in accordance with the invention in the manufacture of a food product. In another aspect, there is provided a use of an expression vector or system in accordance with the invention in the manufacture of a food product.

The present invention also covers using the compounds as a component of pharmaceutical combinations with other components to deliver medical or physiological benefit to the consumer.

Combination with Other Components

Accordingly, the compounds may be used in combination with other components.

Examples of other components include one or more of: thickeners, gelling agents, emulsifiers, binders, crystal modifiers, sweetners (including artificial sweeteners), rheology modifiers, stabilisers, anti-oxidants, dyes, enzymes, carriers, vehicles, excipients, diluents, lubricating agents, flavouring agents, colouring matter, suspending agents, disintegrants, granulation binders etc. These other components may be natural. These other components may be prepared by use of chemical and/or enzymatic techniques.

As used herein the term “thickener or gelling agent” as used herein refers to a product that prevents separation by slowing or preventing the movement of particles, either droplets of immiscible liquids, air or insoluble solids.

The term “stabiliser” as used here is defined as an ingredient or combination of ingredients that keeps a product (e.g. a food product) from changing over time.

The term “emulsifier” as used herein refers to an ingredient (e.g. a food product ingredient) that prevents the separation of emulsions.

As used herein the term “binder” refers to an ingredient (e.g. a food ingredient) that binds the product together through a physical or chemical reaction.

The term “crystal modifier” as used herein refers to an ingredient (e.g. a food ingredient) that affects the crystallisation of either fat or water.

“Carriers” or “vehicles” mean materials suitable for compound administration and include any such material known in the art such as, for example, any liquid, gel, solvent, liquid diluent, solubiliser, or the like, which is non-toxic and which does not interact with any components of the composition in a deleterious manner.

Examples of nutritionally acceptable carriers include, for example, water, salt solutions, alcohol, silicone, waxes, petroleum jelly, vegetable oils, and the like.

Examples of excipients include one or more of: microcrystalline cellulose and other celluloses, lactose, sodium citrate, calcium carbonate, dibasic calcium phosphate, glycine, starch, milk sugar and high molecular weight polyethylene glycols.

Examples of disintegrants include one or more of: starch (preferably corn, potato or tapioca starch), sodium starch glycollate, croscarmellose sodium and certain complex silicates.

Examples of granulation binders include one or more of: polyvinylpyrrolidone, hydroxypropylmethylcellulose (HPMC), hydroxypropylcellulose (HPC), sucrose, maltose, gelatin and acacia.

Examples of lubricating agents include one or more of: magnesium stearate, stearic acid, glyceryl behenate and talc.

Examples of diluents include one or more of: water, ethanol, propylene glycol and glycerin, and combinations thereof.

The other components may be used simultaneously (e.g. when they are in admixture together or even when they are delivered by different routes) or sequentially (e.g. they may be delivered by different routes).

As used herein the term “component suitable for animal or human consumption” means a compound which is or can be added to the composition of the present invention as a supplement which may be of nutritional benefit, a fibre substitute or have a generally beneficial effect to the consumer.

By way of example, the components may be prebiotics such as alginate, xanthan, pectin, locust bean gum (LBG), inulin, guar gum, galacto-oligosaccharide (GOS), fructo-oligosaccharide (FOS), lactosucrose, soybean oligosaccharides, palatinose, isomalto-oligosaccharides, gluco-oligosaccharides and xylo-oligosaccharides.

Food

The compounds may be used as—or in the preparation of—a food. Here, the term “food” is used in a broad sense—and covers food and food products for humans as well as food for animals (i.e. a feed). In a preferred aspect, the food is for human consumption.

The food may be in the form of a solution or as a solid—depending on the use and/or the mode of application and/or the mode of administration

Food Ingredients and Supplements

The compounds may be used as a food ingredient.

As used herein the term “food ingredient” includes a formulation, which is or can be added to functional foods or foodstuffs and includes formulations which can be used at low levels in a wide variety of products that require, for example, acidifying or emulsifying.

The food ingredient may be in the form of a solution or as a solid—depending on the use and/or the mode of application and/or the mode of administration.

The compounds may be—or may be added to—food supplements.

Functional Foods and Nutraceuticals

The compounds may be—or may be added to—functional foods.

As used herein, the term “functional food” means food which is capable of providing not only a nutritional effect and/or a taste satisfaction, but is also capable of delivering a further beneficial effect to consumer.

Although there is no legal definition of a functional food, most of the parties with an interest in this area agree that they are foods marketed as having specific health effects.

Food Products

The compounds of the present invention can be used in the preparation of food products such as one or more of: confectionery products, dairy products, meat products, poultry products, fish products and bakery products.

By way of example, the compounds of the present invention can be used as ingredients to soft drinks, a fruit juice or a beverage comprising whey protein, health teas, cocoa drinks, milk drinks and lactic acid bacteria drinks, yoghurt, drinking yoghurt and wine.

For certain aspects, preferably the foodstuff is a soft drink. For example, the compounds of the present invention may be used as an acidulant to provide tartness and/or to act as a preservative.

For certain aspects, preferably the foodstuff is wine. For example, the compounds of the present invention may promote graceful ageing and crispness of flavour.

For certain aspects, preferably the foodstuff is a bakery product—such as bread, Danish pastry, biscuits or cookies.

For certain aspects, preferably the foodstuff is a confectionery product. By way of example, the composition of the present invention may enhance natural flavouring and/or lower the pH level. Lowering the pH level may inhibit the development of micro-organisms and mould.

The press invention also provides a method of preparing a food or a food ingredient, the method comprising admixing carotenoid cleavage compounds produced by the process of the present invention or the composition according to the present invention with another food ingredient. The method for preparing or a food ingredient is also another aspect of the present invention.

Pharmaceutical

The carotenoid cleavage compounds may also be used as—or in the preparation of—a pharmaceutical. Here, the term “pharmaceutical” is used in a broad sense—and covers pharmaceuticals for humans as well as pharmaceuticals for animals (i.e. veterinary applications). In a preferred aspect, the pharmaceutical is for human use and/or for animal husbandry.

The pharmaceutical can be for therapeutic purposes—which may be curative or palliative or preventative in nature. The pharmaceutical may even be for diagnostic purposes.

When used as—or in the preparation of—a pharmaceutical, the product and/or the compounds of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the from of a solution or as a solid—depending on the use and/or the mode of application and/or the mode of administration.

Pharmaceutical Ingredient

The product and/or the compounds of the present invention may be used as pharmaceutical ingredients. Here, the product and/or the composition of the present invention may be the sole active component or it may be at least one of a number (i.e. 2 or more) active components.

The pharmaceutical ingredient may be in the form of a solution or as a solid—depending on the use and/or the mode of application and/or the mode of administration.

The pharmaceutical ingredient may be in the form of an effervescent product to improve the dissolving properties of the pharmaceutical.

Forms

The product and/or the compounds of the present invention may be used in any suitable form—whether when alone or when present in a composition. Likewise, carotenoid cleavage compounds produced in accordance with the present invention (i.e. ingredients—such as food ingredients, functional food ingredients or pharmaceutical ingredients) may be used in any suitable form.

Suitable examples of forms include one or more of: tablets, pills, capsules, ovules, solutions or suspensions, which may contain flavouring or colouring agents, for immediate-, delayed-, modified-, sustained-, pulsed- or controlled-release applications.

By way of example, if the product and/or the composition are used in a tablet form—such as for use as a functional ingredient—the tablets may also contain one or more of: excipients, disintegrants, granulation binders, or lubricating agents.

Examples of nutritionally acceptable carriers for use in preparing the forms include, for example, water, salt solutions, alcohol, silicone, waxes, petroleum jelly and the like.

Preferred excipients for the forms include lactose, starch, a cellulose, milk sugar or high molecular weight polyethylene glycols.

For aqueous suspensions and/or elixirs, carotenoid cleavage compounds may be combined with various sweetening or flavouring agents, colouring matter or dyes, with emulsifying and/or suspending agents and with diluents such as water, ethanol, propylene glycol and glycerin, and combinations thereof.

The forms may also include gelatin capsules; fibre capsules, fibre tablets etc.

General Recombinant DNA Methodology Techniques

The present invention employs, unless otherwise indicated, conventional techniques of chemistry, molecular biology, microbiology, recombinant DNA and immunology, which are within the capabilities of a person of ordinary ski in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### EXAMPLES

The invention is now further illustrated in the following non-limiting examples.

Important for carotenoid cleavage are genes that are part of a family of CCD (carotenoid cleavage dioxygenase) genes. A mouse gene (less than 30% identical to any plant gene) has been described, which, upon expression in a β-carotene producing bacterium, was shown to produce β ionone (Kiefer et al., 2001). Other CCD genes (either from plant origin or from other organisms) have never been reported to produce α-, β- or pseudo ionone in micro-organisms.

1. Using the CCD Gene from Arabidopsis for Production of β Ionone

The CCD gene from Arabidopsis has been described by Schwartz et al. (2001) J Biol Chem. 276(27):25208-11. To demonstrate its use for producing β ionone, it is cloned as follows.

1.1 RNA Isolation and cDNA Synthesis

Total RNA is isolated from Arabidopsis thaliana Ws using the RNeasy Plant Mini Kit from Qiagen, according to the manufacturer's instructions. 1 ug of total RNA is used in a volume of 3 ul, and mixed with 1 ul polyT primer (10 uM). The mixture is incubated at 70° C. for 2 minutes, and immediately put on ice for 2 minutes. Then 2 ul 5×1st strand buffer (Invitrogen), 1 ul 100 mM DTT, 1 ul 10 mM dNTP, 1 ul Rnasin (Invitrogen) and 1 ul SST Reverse Transcriptase (Invitrogen) are added and the mixture is incubated at 42° C. for 90 minutes. After this, the mixture is inactivated for 7 minutes at 70° C., and stored on ice.

1.2 Amplification of the CCD Coding Region

To amplify the CCD gene, 2 ul of cDNA is used in an amplification reaction mix. The mix further contains 0.5 mM dNTP, 2.5 ul 10×BD Advantage 2 PCR buffer (BD Bioscience), 0.5 ul 50× Advantage 2 Polymerase mix (BD Bioscience) and 0.4 uM of oligonucleotides AtCCDsense and AtCCDanti. The amplification reaction mix is incubated for 5 minutes at 94° C., and subsequently subjected to 30 cycles of 30 seconds 94° C., 30 seconds 50° C. and 3 minutes 72° C. After these cycles, the mixture is incubated at 72° C. for 5 minutes, after which it is cooled to 10° C. The amplified product is purified using the Qiaquick PCR purification kit (Qiagen). The purified fragment (which is about 1700 bp, as analyzed on a 1% agarose gel) is ligated into the pGEM-T easy vector, using the pGEM-T Easy Vector System I (Promega), and subsequently brought into E. coli XL-1 Blue cells by transformation according to standard procedures. Transformed cells are plated on LB-agar plates with 100 ug/ml ampicillin. Of the resulting colonies after overnight incubation at 37° C., three are grown in liquid culture. Clones containing plasmids with inserts are identified by restriction digestion with EcoRI. Plasmid pGEMT-AtCCD#1 is identified in this way.

1.3 Cloning of the CCD Gene into an Expression Vector

To clone the AtCCD cDNA into expression vector pRSETA (Invitrogen), about 1 ug of pGEMT-AtCCD#1 is cleaved with EcoRI and BamHI in buffer React 3 (Invitrogen), in parallel with 1 ug of plasmid pRSETA for 2 hours. Both digestions are loaded on a 1% agarose gel. After electrophoresis, fragments of the expected size (about 1700 bp for the AtCCD fragment and about 2900 bp for the pRSETA vector DNA) are observed, and isolated from the gel using Qiaex II DNA isolation kit (Qiagen). Fragments are brought into 30 ul EB buffer (50 mM Tris pH=8.5). 1 ul of BamHI-EcoRI cleaved pRSETA and 10 ul of EcoRI-BamHI cleaved AtCCD fragment are mixed with 3 ul 5× ligase buffer (Invitrogen) and 1 ul of T4 ligase (Invitrogen). The ligation mixture is incubated for 3 hours at 16° C. and 10 ul of it is transformed into competent E. coli XL-1 blue by standard procedures. The transformation mixture is plated on petridishes containing LB medium, 1.5% technical agar and 100 ug/ml ampicillin. After overnight incubation at 37° C., colonies are picked into 3 ml liquid LB medium with 100 ug/ml ampicillin and grown overnight at 37° C. shaking at 250 rpm. Plasmid is isolated from 1.5 ml of this culture using the Qiagen plasmid isolation kit, and clones containing plasmids with inserts are identified by restriction digestion with EcoRI and BamHI. Plasmid pRSETA-AtCCD#1 is identified in this way.

1.4 The Nucleotide and Protein Sequence of AtCCD

The nucleotide sequence of the inserted DNA fragment of pRSETA-AtCCD#1 is analyzed using oligonucleotides T7, pRSETrev and AtCCDs791 and AtCCDa895. The nucleotide sequence and the encoded protein sequence of AtCCD are represented below, in the section ‘Sequence information’. It contains 1617 nucleotides, including the start- and stop codons, and encodes a 538 amino acid protein. The encoded AtCCD protein is 99% identical (533 out of 538 residues) to accession gi|3096910|emb|CAA06712.1|.

1.5 Combining AtCCD with a Micro-Organism that Produces β Carotene

To assess the potential of AtCCD to produce β ionone, it is brought into a host cell producing T7 polymerase and β carotene. The T7 polymerase starts transcription from the T7 promoter, which is located just upstream of the AtCCD cDNA in plasmid pRSETA-AtCCD#1. E. coli stain BL21 is able to produce T7 polymerase in the absence of glucose. When this strain carries plasmid pRSETA-AtCCD#1, the AtCCD protein is produced. β carotene can be produced in E. coli by providing it with the plasmid pAC-BETA, which has been described by Cunningham et al., (1996) Plant Cell 8, 1613-1626.

To construct bacteria that are capable of producing both β carotene and AtCCD enzyme, E. coli BL21 CodonPlus-RIL (Stratagene) competent cells are transformed with pAC-BETA according to the manufacturer's instructions. Recombinant E. coli are selected overnight at 37° C. on LB-agar plates with 1% glucose and 30 ug/ml chloramphenicol. A colony of E. coli BL21 with pAC-BETA is inoculated in 1 ml LB 1775 with chloramphenicol and glucose and the culture is grown overnight at 250 rpm and 37° C. The BL21-pAC-BETA is made competent by diluting the overnight culture 100-fold in fresh LB medium with 1% glucose, and shaking it at 37° C. until an optical density at 600 nm of 0.4 is reached. 10 ml of culture is centrifuged for 5 minutes at 400×g. Supernatant is discarded and replaced by 10 ml of an ice-cold solution of 10 mM CaCl₂ and 1 mM Tris-HCl pH=7.5. Cells are resuspended and immediately centrifuged again at 400×g for 5 minutes. After discarding the supernatant, cells are resuspended in 2 ml of an ice-cold solution of 75 mM CaCl₂ and 1 mM Tris-HCl pH=7.5. After incubation on ice for at least 30 minutes, cells are used for plasmid transformation by standard procedures. Plasmids pRSETA and pRSETA-AtCCD#1 are used to transform these cells, and transformed colonies are selected on LB-agar plates supplied with 1% glucose, 20 ug/ml chloramphenicol and 50 ug/ml ampicillin. The bacteria in the resulting colonies are referred to as BL21-pAC-BETA-pRSETA and BL21-pAC-BETA-pRSETA-AtCCD, respectively.

1.6 Production of β Ionone

To produce β ionone, colonies of BL21-pAC-BETA-pRSETA and BL21-pAC-BETA-pRSETA-AtCCD are transferred to 1 ml liquid LB supplied with 1% glucose, 20 ug/ml chloramphenicol and 100 ug/ml ampicillin and grown overnight at 30° C. and 250 rpm. The next day, 0.5 ml of the overnight culture is used to inoculate a 2-liter erlenmeyer with 50 ml fresh LB medium with 20 ug/ml chloramphenicol and 50 ug/ml ampicillin. This culture is shaken at 250 rpm and 28° C. for 3 hours, and then further shaken at 250 rpm and 18° C. for 48 hours in the dark

1.7 Extraction and Analysis of β Ionone

To isolate the β ionone, the total culture is brought into a separating funnel. To 50 ml of the culture, 20 ml of pentane:ether (80:20) is added, and the culture is vigorously mixed for at least 20 seconds, after which it is allowed to stand for 5 minutes to allow phases to separate. The lower phase is discarded, while the upper (organic) phase, which is quite viscous, is collected in centrifuge tubes. The organic phase is centrifuged for 5 minutes at 1200×g. After centrifugation, the upper phase will also comprise a gel-like substance, which can be removed by disturbing it with a glass bar. The clear part of the upper phase is collected in a glass tube, and pentane:ether is aspired from it under a nitrogen flow. The β ionone will be in the residue at the bottom of the tube.

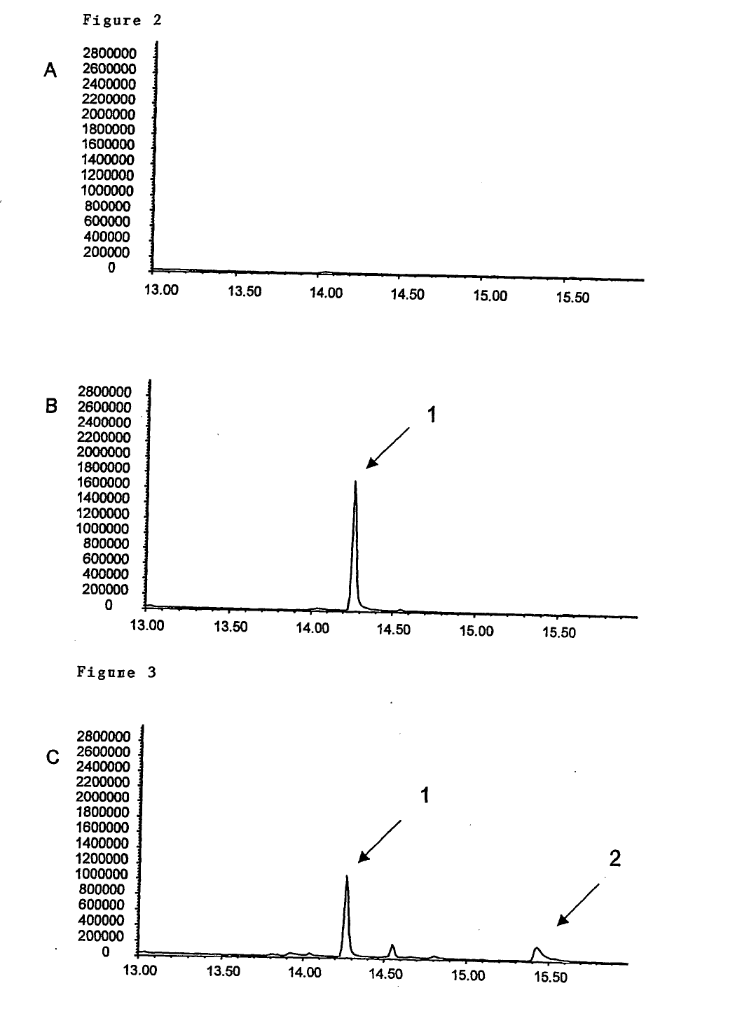

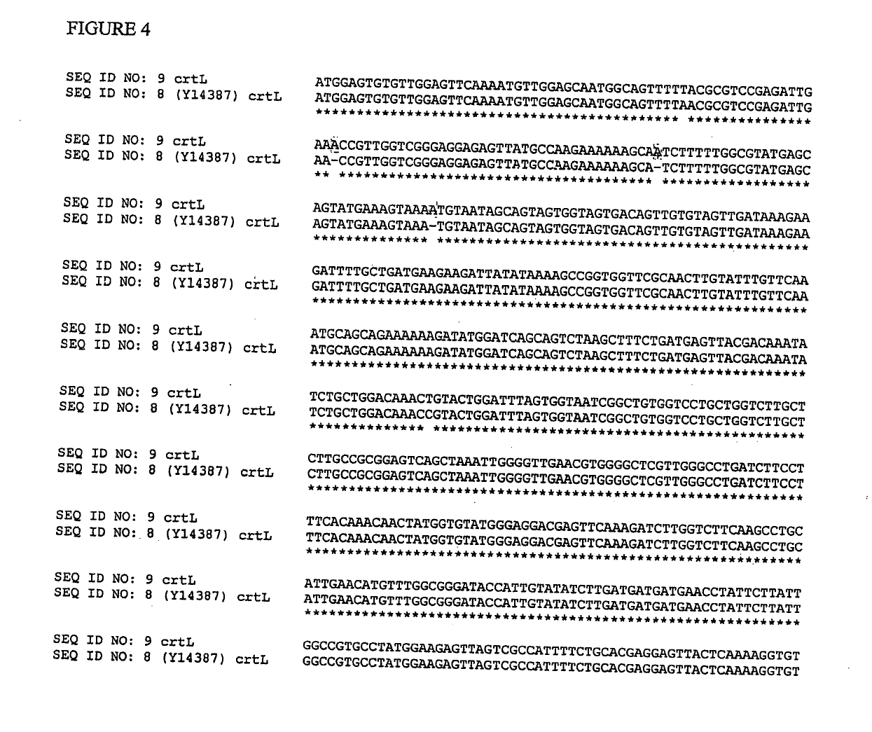

To identify and quantify the β ionone, the residue is dissolved in 500 ul pentane ether, from which 2 μL is analyzed by GC-MS using a gas chromatograph (5890 series II, Hewlett-Packard) equipped with a 30-m×0.25-mm i.d., 0.25-μm film thickness column (5MS, Hewlett-Packard) and a mass-selective detector (model 5972A, Hewlett-Packard). The GC is programmed at an initial temperature of 45° C. for 1 min, with a ramp of 10° C. min⁻¹ to 280° C. and final time of 2.5 min. The injection port (splitless mode), interface, and MS source temperatures are 250° C., 290° C., and 180° C., respectively, and the He inlet pressure is controlled with an electronic pressure control to achieve a constant column flow of 1.0 mL min⁻¹. The ionization potential of the MS is set at 70 eV. Compounds are detected by the MS in the scan mode, starting from 5 minutes after injection. A standard of β ionone (0.2 ug/ml) is injected after the analysis of samples from BL21-pAC-BETA-pRSETA and BL21-pAC-BETA-pRSETA-AtCCD extracts. β ionone is detected at 14.3 minutes, and shows a characteristic spectrum with a dominant mass peak at m/z 177. Upon quantification, the BL21-pAC-BETA-pRSETA appears to have produced no β-ionone (FIG. 2A), while BL21-pAC-BETA-pRSETA-AtCCD has produced 50 ug β ionone per 50 ml of culture (FIG. 2B).

2. Using the CCD Gene from Rubus idaeus for Production of β Ionone and Pseudo Ionone

The CCD gene from raspberry (Rubus idaeus) is involved in producing compounds that determine the flavor of the raspberry fruit. The gene encoding the raspberry CCD has not been described previously.

2.1 RNA Isolation and cDNA Synthesis

To isolate the gene for ionone formation from raspberry, total RNA is isolated from ripe raspberries from the cultivar Tulameen. For this purpose, 5 gram raspberries is frozen in liquid nitrogen and ground to powder using a coffee-grinder which is pre-cooled with liquid nitrogen. The powder is immediately transferred to a 250 ml centrifuge tube containing 50 ml extraction buffer (2% CTAB, 100 mM Tris pH8.2, 1.4 M NaCl, 20 mM EDTA) pre-warmed to 65° C., and 50 ul 2-mercaptoethanol is added. The tube is incubated at 65° C. for an hour and is agitated every 10 minutes. The tube is transferred to room temperature and left to stand until the tube is no longer warm. Then 50 ml chloroform/isoamylalcohol (24:1) is added, the mixture is vigorously agitated for 5 seconds and centrifuged at 12,000×g for 15 minutes at room temperature. The watery phase is transferred into another tube, to which 50 ml of chloroform/isoamylalcohol (24:1) is added. The mixture is vigorously agitated for 5 seconds and centrifuged at 12,000×g for 15 minutes at room temperature. The watery phase is transferred to another tube, to which 33 ml of 10 M LiCl is added. After careful mixing, the tube is put at 4° C. and left overnight. The next day, the tube is centrifuged at 20,000×g for 20 minutes at 4° C., and supernatant is completely removed. The pellet is solved in 1 ml sterile T2 buffer, and extracted with 1 ml phenol, phenol/chloroform/isoamylalcohol (24:24:1) and chloroform/isoamylalcohol (24:1). To the final water phase, 0.11 volume of 3M sodium acetate and 3 volumes of 100% ethanol are added, and the mixture is incubated overnight at −70° C. The next day the tube is centrifuged for 15 minutes at 14,000×g at 4° C. The supernatant is removed, the pellet washed with 70% ethanol and air-dried for 5 minutes. The pellet is dissolved in 20 ul water, and the concentration of RNA is measured by diluting this solution in water, and measuring the absorption at 260 nm and 280 nm.

To synthesize cDNA, 1 ug of raspberry RNA is used in a volume of 3 ul, and mixed with 1 ul polyT primer (10 up. The mixture is incubated at 70° C. for 2 minutes, and immediately put on ice for 2 minutes. Then 2 ul 5×1st strand buffer (Invitrogen), 1 ul 100 mM DTT, 1 ul 10 mM dNTP, 1 ul Rnasin (Invitrogen) and 1 ul SST Reverse Transcriptase (Invitrogen) are added and the mixture is incubated at 42° C. for 90 minutes. After this, the mixture is inactivated for 7 minutes at 70° C., and stored on ice.

2.2 Amplifying a Fragment from the Raspberry CCD Gene

To amplify a fragment of a CCD cDNA, a 2-step amplification protocol is used. For the First amplification, 2 ul of cDNA is used in 25 μl amplification reaction mix. The mix further contains 0.5 mM dNTP, 2.5 ul 10×BD Advantage 2 PCR buffer (BD Bioscience), 0.5 ul 50× Advantage 2 polymerase mix (BD Bioscience) and 0.4 uM of oligonucleotides CCDfwd1 and CCDrev2. The amplification reaction mix is incubated for 5 minutes at 94° C., and subsequently subjected to 10 cycles of 30 seconds 94° C., 30 seconds 40° C. and 3 minutes 72° C., then 10 cycles of 30 seconds 94° C., 30 seconds 45° C. and 3 minutes 72° C., and finally 10 cycles of 30 seconds 94° C., 30 seconds 50° C. and 3 minutes 72° C. After these cycles, the mixture is incubated at 72° C. for 5 minutes, after which it is cooled to 10° C. 1 ul of this First PCR reaction is used in a Second Reaction, where it is mixed with 0.5 mM dNTP, 2.5 ul 10×BD Advantage 2 PCR buffer (BD Bioscience), 0.5 ul 50× Advantage 2 polymerase mix (BD Bioscience) and 0.4 uM of oligonucleotides CCDfwd2 and CCDrev1, in a volume of 25 ul. The same amplification procedure as described for the First amplification is carried out. When analyzed on gel, the resulting PCR product from the second amplification should be about 550 bp in size. The amplified product is purified using the Qiaquick PCR purification kit (Qiagen). The purified fragment is ligated into the pGEM-T easy vector, using the pGEM-T Easy Vector System I (Promega), and subsequently brought into E. coli XL-1 Blue cells by transformation according to standard procedures. Transformed cells are plated on LB-agar plates with 100 ug/ml ampicillin. Of the resulting colonies after overnight incubation at 37° C., three are grown in liquid culture. Clones containing plasmids with inserts are 1895 identified by restriction digestion with EcoRI. Plasmid pGEMT-RiCCD#1 is identified in this way. Sequence analysis of this fragment using oligonucleotides T7 and SP6 reveals a sequence overlapping with nucleotides 366 to 852 from RiCCD as in the section ‘Sequence information’.

2.3 Amplifying CCD cDNA Ends

To extend this fragment to the 3′ end and 5′ end of the cDNA, the SMART RACE cDNA Amplification Kit (Clontech) is applied. Firstly, based on the pGEMT-RiCCD#1 DNA sequence, two forward primers (RiCCDfwd1 and RiCCDfwd2) and two reverse primers (RiCCDrev1 and RiCCDrev2) are designed. Total RNA from red Tulameen raspberries is isolated as described above. To obtain 5′ RACE cDNA, 3 ul of this RNA is mixed with 1 ul of oligonucleotide PolyT (10 uM) and 1 ul SMART IIA oligonucleotide (10 uM). To obtain 3′ RACE cDNA, 3 ul of this RNA is mixed with 1 ul 3′CDS primer (10 uM). Both mixtures are heated for 2 minutes at 70° C., cooled on ice for 2 minutes and mixed with 2 ul 5× First-Stand Buffer, 1 ul 20 mM DTT, 1 ul 10 mM dNTP and 1 ul Powerscript Reverse Transcriptase. For cDNA synthesis, the mixtures are incubated on 42° C. for 1.5 hour. After that, 100 ul Dilution buffer (10 mM Tricine KOH pH=8.5+1 mM EDTA) is added and the mixtures are incubated at 70° C. for 7 minutes, and cooled on ice. For the 5′ RACE reaction, 2.5 ul 5′RACE cDNA is mixed with 1 ul RiCCDrev1 (10 uM), 34.5 ul water, 5 ul 10× Advantage 2 PCR buffer, 1 ul 10 mM dNTP and 5 ul UPM (consisting of 0.4 uM UPM Long oligonucleotide and 2 uM UPM Short oligonucleotide). For the 3′RACE reaction, 2.5 ul 3′RACE cDNA is mixed with 1 ul RiCCDfwd1 (10 uM), 34.5 ul water, 5 ul 10× Advantage 2 PCR buffer, 1 ul 10 mM dNTP and 5 μl UPM (consisting of 0.4 uM UPM Long oligonucleotide and 2 uM UPM Short oligonucleotide). These RACE mixes are both incubated for 5 minutes at 94° C., and subsequently subjected to 25 cycles of 30 seconds 94° C., 30 seconds 50° C. and 3 minutes 72° C. After these cycles, both the mixtures are incubated on 68° C. for 5 minutes, after which they are cooled to 10° C. Both the 5′RACE mix and the 3′RACE mix are diluted 50 times in Dilution buffer. Of the 5′RACE dilution, 5 ul is mixed with 34.5 ul water, 5 ul 10× Advantage 2 PCR buffer, 1 ul 10 mM dNTP 1 ul of oligonucleotide RiCCDrev2 and 1 ul of oligonucleotide NUP. Of the 3′RACE dilution, 5 ul is mixed with 34.5 ul water, 5 ul 10× Advantage 2 PCR buffer, 1 ul 10 mM dNTP 1 ul of oligonucleotide RiCCDfwd2 and 1 ul of oligonucleotide NUP. Both mixtures are incubated for 5 minutes at 94° C., and subsequently subjected to 20 cycles of 30 seconds 94° C., 30 seconds 50° C. and 3 minutes 68° C. After these cycles, the amplification mixtures are incubated on 68° C. for 5 minutes, after which they are cooled to 10° C. Both mixtures are analyzed on a 1% agarose gel. The 5′ RACE amplification mixture contains a 550 bp fragment, while the 3′RACE amplification mixture contains a 1100 bp fragment.

Both fragments are purified from gel using the QiaEX II kit (Qiagen). Each of the purified fragments is ligated into the pGEM-T easy vector, using the pGEM-T Easy Vector System I (Promega), and subsequently brought into E. coli XL-1 Blue cells by transformation according to standard procedures. Transformed cells are plated on LB-agar plates with 100 ug/ml ampicillin, and grown overnight at 37° C. Clones with an inset from the expected size are identified by growing them in liquid culture, isolating plasmid from it and digesting the plasmid with restriction enzyme EcoRI according to standard procedures.

2.4 Analysing the Sequence of the Raspberry CCD Gene (RiCCD)

From both the 3′RACE experiment and the 5′RACE experiment, plasmids with an insert of the expected size (550 bp for 5′RACE, 1100 bp for 3′RACE) are analyzed by sequencing them with oligonucleotides T7 and SP6. The 3′ RACE plasmids are further analyzed with oligonucleotide RiCCDint1 and RiCCDint2.

The resulting sequence data from both pGEMT-RiCCD#1, the 5′RACE product and the 3′RACE product are analyzed by BLASE and appear to match plant CCD genes. DNA sequences are assembled using the SEQMAN module of the Lasergene system (DNASTAR). A consensus sequence is assembled and corrected for obvious interpretation mistakes. The resulting DNA sequence and translated protein sequence are shown in the section ‘Sequence information’. The RiCCD open reading frame comprises 1696 DNA residues, including start- and stop codon, encoding a protein of 558 amino acids. Analysis by BLASTX reveals that the encoded protein from RiCCD is 80% identical (444 out of 558 amino acids from RiCCD) to nine-cis-epoxycarotenoid dioxygenase 1 from Pisum sativum (accession gi|22335695|dbj|BAC10549.1|), being the closest homologue in the database.

2.5 Cloning the RiCCD Gene into an Expression Vector

The coding region from the RiCCD cDNA is amplified from raspberry cDNA by taking 2 ul of the raspberry cDNA described above, and mixing it with 0.5 mM dNTP, 2.5 ul 10×BD Advantage 2 PCR buffer (BD Bioscience), 0.5 ul 50× Advantage 2 polymerase mix (BD Bioscience) and 0.4 uM of oligonucleotides RiCCDstart and RiCCDend. The amplification reaction mix is incubated for 5 minutes at 94° C., and subsequently subjected to 10 cycles of 30 seconds 94° C., 30 seconds 50° C. and 3 minutes 72° C., and 20 cycles of 30 seconds 94° C., 30 seconds 55° C. and 3 minutes 72° C. After these cycles, the mixture is incubated on 72° C. for 5 minutes, after which it is cooled to 10° C. The amplified product is purified using the Qiaquick PCR purification kit (Qiagen). The purified fragment (which is about 1600 bp, as analyzed on a 1% agarose gel) is ligated into the pGEM-T easy vector, using the pGEM-T Easy Vector System I (Promega), and subsequently brought into E. coli XL-1 Blue cells by transformation according to standard procedures. Transformed cells are plated on LB-agar plates with 100 ug/ml ampicillin. Of the resulting colonies after overnight incubation at 37° C., three are grown in liquid culture. Clones containing plasmids with inserts are identified by restriction digestion with EcoRI. Plasmid pGEMT-RiCCD#2 is identified in this way.

About 1 ug of pGEMT-RiCCD#2 is cleaved with KpnI and BamHI in buffer React 3 (Invitrogen), in parallel with 1 ug of plasmid pRSETA. Both digestions are loaded on a 1% agarose gel. After electrophoresis, fragments of the expected size (about 1600 bp for the RiCCD fragment and about 2900 bp for the vector DNA) are observed, and isolated from the gel using Qiaex II DNA isolation kit (Qiagen). Fragments are brought into 30 ul EB buffer (50 mM Tris pH=8.5). To clone the RiCCDgene from raspberry into pRSETA, 1 ul of BamHI-KpnI cleaved pRSETA and 10 ul of purified and cleaved RiCCD product are mixed with 3 ul 5× ligase buffer (Invitrogen) and 1 ul of T4 ligase (Invitrogen). The ligation mixture is incubated for 3 hours at 16° C. and 10 ul of it is transformed into competent E. coli XL-1 blue by standard procedures. The transformation mixture is plated on 25 ml petridishes containing LB medium, 1.5% technical agar and 100 ug/ml ampicillin. After overnight incubation at 37° C., colonies are picked into 3 ml liquid LB medium with 100 ug/ml ampicillin and grown overnight at 37° C. shaking at 250 rpm. Plasmid is isolated from 1.5 ml of this culture using the Qiagen plasmid isolation kit, and clones containing plasmids with inserts are identified by restriction digestion with KpnI and Bane. Plasmid pRSETA-RiCCD#1 is identified in this way. The nucleotide sequence of the inserted DNA fragment of pRSET-RiCCD#1 is analyzed using oligonucleotides 17 and PRSETrev. A sequence alignment with the DNA sequence obtained for pGEMT-RiCCD#2 reveals that both RiCCD coding sequences are identical.

2.6 Use of RiCCD for Production of β Ionone and Pseudo Ionone

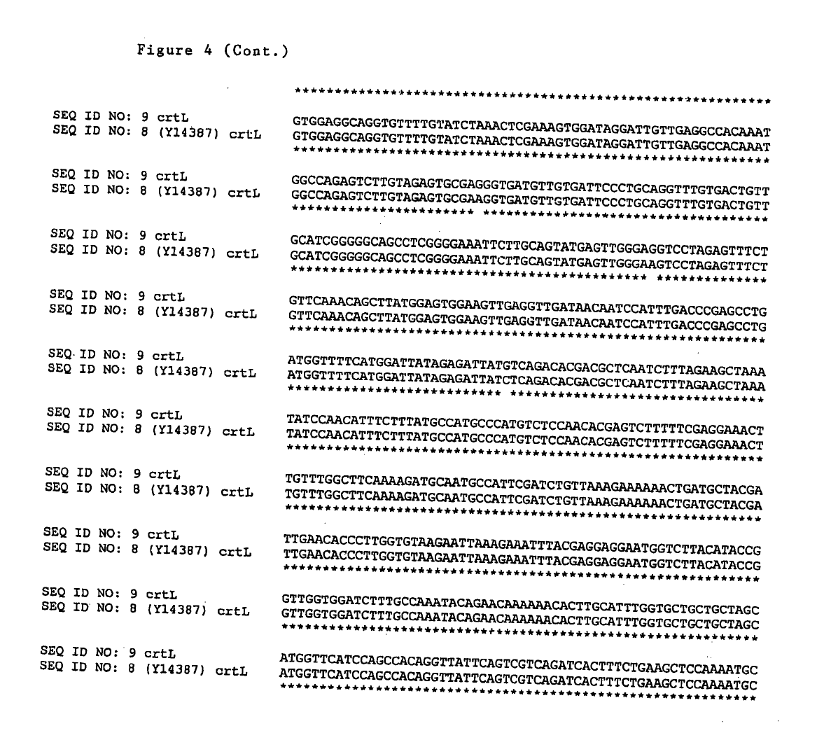

The pRSET-RiCCD#1 plasmid is brought into bacteria producing β carotene, in a comparable way as described in section 1.5. Subsequently, production of ionone is performed as described in section 1.6. Analysis of the products is performed as described in section 1.7. The pRSET-RiCCD#1 plasmid has resulted in production of β-ionone, (about 30 ug per 50 ml of culture) but in addition, about 20 ug of E,E pseudo ionone is observed in the exact (FIG. 3). This pseudo ionone is derived from lycopene by cleavage by RiCCD. Lycopene is made transiently in the E. coli, as an intermediate in the synthesis of β ionone. Alternatively, lycopene can be made by a strain harbouring plasmid pAC-Lyco-1 (see below).

The CCD Gene from Zea mais

3.1 RNA Extraction and cDNA Synthesis

RNA from roots of corn (Zea mais) cultivar Dent MBS847 is extracted by the SV total RNA Isolation kit from Promega, according to the manufacturer's instructions. This RNA is transcribed into cDNA as described in section 1.1.

3.2 Amplification of the Corn CCD Gene

The CCD protein from Zea mais (ZmCCD) is encoded by accession TC220599 in the TIGR database. To amplify the ZmCCD gene, 2 ul of cDNA is used in an amplification reaction mix. The mix fixer contains 0.5 mM DNTP, 2.5 ul 10×BD Advantage 2 PCR buffer (BD Bioscience), 0.5 ul 50× Advantage 2 polymerase mix (BD Bioscience) and 0.4 uM of oligonucleotides ZmCCDfwd1 and ZmCCDrev1. The amplification reaction mix is incubated for 5 minutes at 94° C., and subsequently subjected to 30 cycles of 30 seconds 94° C., 30 seconds 55° C. and 3 minutes 72° C. After these cycles, the mixture is incubated at 72° C. for 5 minutes, after which it is cooled to 10° C. 1 ul of this reaction is used as a template for a second PCR reaction, under the same conditions, but with oligonucleotides ZmCCDfwd2 and ZmCCDrev2. The product that is found, of about 1650 bp in size, is cloned into pGEMTeasy as described in section 1.2, to yield plasmid pGEMT-ZmCCD#1 and this plasmid is sequenced using oligonucleotides 17, SP6, ZmCCDint1 and ZmCCDint2. The sequence of the ZmCCD coding region, and the encoded protein, is provided in the Sequence information section (below).

3.3 Cloning of ZmCCD into an Expression Vector

One ug of plasmid DNA from pGEMT-ZmCCD#1 and from pRSETA is digested with BamHI and EcoRI in the appropriate buffer. Both digestions are loaded on a 1% agarose gel. After electrophoresis, fragments of the expected size (about 1650 bp for the ZmCCD fragment and about 2900 bp for the vector DNA) are observed, and isolated from the gel using Qiaex II DNA isolation kit (Qiagen). Fragments are brought into 30 ul EB buffer (50 mM Tris pH=8.5). To clone the ZmCCDgene from raspberry into pRSETA, 1 ul of BamHI-EcoRI cleaved pRSETA and 10 ul of purified and cleaved ZmCCD product are mixed with 3 ul 5× ligase buffer (Invitrogen) and 1 ul of T4 ligase (Invitrogen). The ligation mixture is incubated for 3 hours at 16° C. and 10 μl of it is used for transformation of competent E. coli XL-1 blue by standard procedures. The transformation mixture is plated on 25 ml petridishes containing LB medium, 1.5% technical agar and 100 ug/ml ampicillin. After overnight incubation at 37° C., colonies are picked into 3 ml liquid LB medium with 100 ug/ml ampicillin and grown overnight at 37° C. shaking at 250 rpm. Plasmid is isolated from 1.5 ml of this culture using the Qiagen plasmid isolation kit, and clones containing plasmids with inserts are identified by restriction digestion with KpnI and BamHI. Plasmid pRSETA-ZmCCD#1 is identified in this way. The nucleotide sequence of the inserted DNA fragment of pRSET-ZmCCD#1 is analyzed using oligonucleotides 17 and PRSETrev. A sequence alignment with the DNA sequence obtained for pGEMT-ZmCCD#2 reveals that both ZmCCD coding sequences are identical.

3.4 Use of ZmCCD for Production of β ionone

The pRSET-ZmCCD#1 plasmid is brought into bacteria producing β carotene, in a comparable way as described in section 1.5. Subsequently, production of ionone is performed as described in section 1.6. Analysis of the products is performed as described in section 1.7. The presence of the pRSET-ZmCCD#1 plasmid has resulted in production of β-ionone, though it is less than 0.3 ug per 50 ml of culture.

3.5 Quantification of Carotenoid

To assess the efficiency of carotenoid cleavage by the various CCD genes, not only the product (i.e. beta ionone) was quantified, but also the amount of carotenoid which was not cleaved. For this purpose, bacteria which had been used for production of beta ionone, as described in sections 1.6 and 2.6, were also analyzed for carotenoid content. The bacteria of 25 ml culture were centrifuged for 10 minutes at 10.000×g, and the supernatant was removed carefully. Subsequently, carotenoid was extracted from the bacteria and analyzed as described by Bino et al., 2005, New Phytologist 166 (2), 427-438. The results, shown in Table 1 (values are expressed as microgram per 25 ml culture), demonstrate that RiCCD is more efficient in the cleavage of carotenoids than is AtCCD, and much more efficient than ZmCCD. This indicates that the RiCCD enzyme may be useful under circumstances that AtCCD is not sufficiently active.

TABLE 1

| | Beta | Beta | |
|---|---|---|---|
| Strain | carotene | ionone | Pseudo ionone |
| BL21-pAC-BETA-pRSETA | 38.6 | 0 | 0 |
| BL21-pAC-BETA-pRSETA-AtCCD | 7.3 | 24.9 | 0.24 |
| BL21-pAC-BETA-pRSETA-RiCCD | 2.5 | 15.0 | 8.7 |
| BL21-pAC-BETA-pRSETA-ZmCCD | 38.8 | trace | 0 |

4 Production of Alpha Ionone

Alpha ionone can be derived from delta carotene or alpha carotene. Both compounds are derived from lycopene by the action of epsilon cyclase. According to Cunningham et al., 1996, a micro-organism that produces lycopene and expresses an epsilon lycopene cyclase (from Arabidopsis in their case) will produce mainly delta carotene. When such a micro-organism is combined with one of the above described CCD genes, it will produce alpha ionone.

4.1 A Micro-Organism that Produces Lycopene

To obtain pAC-Lyco-1, the plasmid pAC-BETA is subjected to targeted mutagenesis using oligonucleotides CrtYmutS and CrtYmutA, using the QuickChange Site-Directed Mutagenesis kit from Stratagene, and the Phusion high-fidelity polymerase from Finnzymes. The mutations designed in these oligonucleotides are directed to introduce two consecutive stop-codons (TAG-TAA), by changing two nucleotides into adenine (underlined in the section Sequence information, below). These mutations cause the translation of the crtY gene (lycopene cyclase) to end prematurely, producing only a truncated version of the crtY protein (normally 386 amino acids), consisting of only the 43 N-terminal residues. The resulting plasmid, pAC-Lyco-1 is selected by transferring it to E. coli and selecting the transformed cells on plates containing 30 ug/ml chloramphenicol. Sequence analysis using oligonucleotide CrtYseq confirms the correct introduction of the mutations. An analysis of the carotenoids produced by a bacterial strain carrying this plasmid, according to Cunningham et al., (1996), conforms that all-trans lycopene is the major carotenoid produced, with some traces of phytoene, the lycopene precursor, while no beta carotene is observed.

4.2 Cloning of Epsilon Cyclase from Tomato

The epsilon cyclase gene (CrtL-e) from tomato has been described by Ronen et al. (1999) Plant J. 17, 341-351. Its sequence is available as accession Y14387 in the database (see also section Sequence information, SEQ ID NO:8 below).

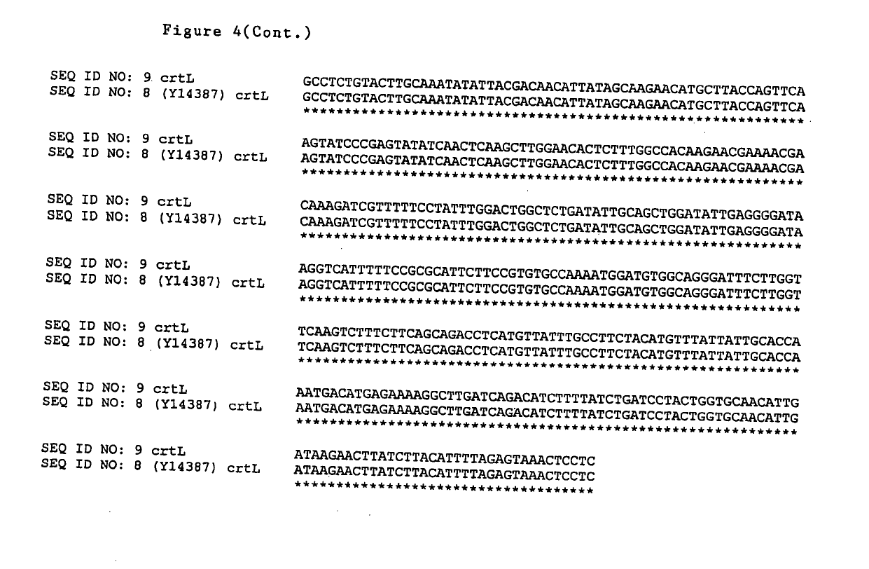

Tomato total RNA is isolated from tomato leaf (cv. Moneymaker) using the RNeasy Plant Mini Kit from Qiagen, according to the manufacturers instructions. The total RNA is converted to cDNA by using procedures described in the SMART RACE cDNA amplification kit as described in section 2.3. Subsequently, the cDNA is used for amplification of the epsilon cyclase open reading frame. To amplify the CrtL-e gene, 2 ul of cDNA is used in an amplification reaction mix. The mix further contains 0.5 mM dNTP, 2.5 ul 10×BD Advantage 2 PCR buffer (BD Bioscience), 0.5 ul 50× Advantage 2 polymerase mix (BD Bioscience) and 0.4 uM of oligonucleotides Dan3 and Dan 4. The amplification reaction mix was subjected to 20 cycles of 30 seconds 94° C., 30 seconds 68° C., and 3 minutes 72° C., and subsequently 15 cycles of 30 seconds 94° C., 30 seconds 60° C., and 3 minutes 72° C. The product of this amplification is not visible on gel, but 3 ul is used as a template in an amplification reaction which further contains 0.5 mM dNTP, 2.5 ul 10×BD Advantage 2 PCR buffer (BD Bioscience), 0.5 ul 50× Advantage 2 polymerase mix (BD Bioscience) and 0.4 uM of oligonucleotides, CrtLfwd and CrtLrev. The amplification reaction mix is incubated with the same procedure as described for oligonucleotides Dan3 and Dan4. The amplified product is purified using the Qiaquick PCR purification kit (Qiagen). The purified fragment (which is about 1700 bp, as analyzed on a 1% agarose gel) is ligated into the pGEM-T easy vector, using the pGEM-T Easy Vector System I (Promega), and subsequently brought into E. coli XL-1 Blue cells by transformation according to standard procedures. Transformed cells are plated on LB-agar plates with 100 ug/ml ampicillin. Of the resulting colonies after overnight incubation at 37° C., three are grown in liquid culture. Clones containing plasmids with inserts are identified by restriction digestion with EcoRI. Plasmid pGEMT-CrtLE#1 is identified in this way. PGEMT-CrtLE#1 is sequenced by using oligonucleotides CrtLfwd, CrtLrev, Dan5, and Dan6. The CrtL sequence obtained is set out in SEQ ID NO:9. The sequence differs to the sequence set out in accession Y14387 (SEQ ID NO:8 herein). FIG. 4 shows an alignment of Y14387 to SEQ ID NO:9 and highlights the differences. In particular, at position 63, 102 and 135 (marked in yellow) there are inserted nucleotides. These indicate that the protein produced from the crtL gene described in SEQ ID NO: 9 would differ from the published protein, especially between amino-acids 21 and 45. SEQ ID NO: 10 sets out the corresponding protein sequence for the CrtL sequence identified.

4.3 Cloning of CrtL-e in an Expression Plasmid

The plasmid pCDF-Duet-1 (Novagen) allows protein expression from a 17 promoter. The plasmid has a CloDF13 origin of replication and is selectable by streptomycin. Therefore it can co-exist with pAC-Lyco-1 and pUC-derived plasmids in a single E. coli cell.

1 ug of pCDF-Duet-1 and pGEMT-CrtLE#1 are digested with restriction enzymes BamHI and SalI in the appropriate buffer, and after separation on a 1% agarose gel, fragments of the predicted length (CrtLE+/−1650 bp, pCDF-Duet-1 (+/−3750 bp) are isolated from gel, and ligated as described in section 1.3. After transformation to E. coli XL-1 blue according to standard procedures, recombinant colonies are selected on LB-agar plates containing streptomycin (50 ug/ml). After confirmation of the nature of the insert by restriction analysis, the plasmid is called pCDF-CrtL-e#1.

4.4 Construction of a Bacterial Strain Capable of Forming Alfa Ionone

To construct bacteria which are capable to produce both delta carotene and AtCCD enzyme, E. coli BL21 CodonPlus-RIL (Stratagene) competent cells are transformed with pAC-Lyco-1 according to the manufacturer's instructions. Recombinant E. coli are selected overnight at 37° C. on LB-agar plates with 1% glucose and 30 ug/ml chloramphenicol. A colony of E. coli BL21 with pAC-Lyco-1 is inoculated in 1 ml LB with chloramphenicol and glucose and the culture is grown overnight at 250 rpm and 37° C. The BL21-pAC-Lyco-1 is made competent by diluting the overnight culture 100-fold in fresh LB medium with 1% glucose until and shaking it at 37° C. until an optical density at 600 nm of 0.4 is reaches 10 ml of culture is centrifuged for 5 minutes at 400×g. Supernatant is discarded and replaced by 10 ml of an ice-cold solution of 10 mM CaCl₂ and 1 mM Tris-HCl pH=7.5. Cells are resuspended and immediately centrifuged again at 400×g for 5 minutes. After discarding the supernatant, cells are resuspended in 2 ml of an ice-cold solution of 75 mM CaCl2 and 1 mM Tris-HCl pH=7.5. After incubation on ice for at least 30 minutes, cells are used for plasmid transformation by standard procedures. Plasmid pCDF-CrtL-e#1 is used to transform these cells, and transformed colonies are selected on LB-agar plates supplied with 1% glucose, 30 ug/ml chloramphenicol and 50 ug/ml streptomycin. The bacteria in the resulting colonies are referred to as BL21-pAC-Lyco-pCDF-CrtL.

A colony of these bacteria is inoculated in 1 ml LB with chloramphenicol, streptomycin and glucose and the culture is grown overnight at 250 rpm and 37° C. The BL21-pAC-Lyco-pCDF-CrtL is made competent by diluting the overnight culture 100-fold in fresh LB medium with 1% glucose, and shaking it at 37° C. until an optical density at 600 nm of 0.4 is reached. 10 ml of culture is centrifuged for 5 minutes at 400×g. Supernatant is discarded and replaced by 10 ml of an ice-cold solution of 10 mM CaCl₂ and 1 mM Tris-HCl pH=7.5. Cells are resuspended and immediately centrifuged again at 400×g for 5 minutes. After discarding the supernatant, cells are resuspended in 2 ml of an ice-cold solution of 75 mM CaCl2 and 1 mM Tris-HCl pH=7.5. After incubation on ice for at least 30 minutes, cells are used for plasmid transformation by standard procedures. Plasmid pRSETA-RiCCD#1 (or pRSETA-AtCCD#1 or pRSETA-ZmCCD#1) is used to transform these cells, and transformed colonies are selected on LB-agar plates supplied with 1% glucose, 30 ug/ml chloramphenicol, 100 ug/ml ampicillin and 50 ug/ml streptomycin. The bacteria in the resulting colonies are referred to as BL21-pAC-Lyco-pCDF-CrtL-pRSETA-RiCCD.

4.5 Production of Alfa Ionone

Production of alfa ionone using BL21-pAC-Lyco-pCDF-CrL-pRSETA-RiCCD bacteria is performed as described in section 1.6. Analysis of the products is performed as described in section 1.7, using an authentic alfa-ionone standard. The strain has produced alfa-ionone to about 1 ug per 50 ml of culture.

4.6 Construction of a Bacterial Strain Capable of Forming Pseudo Ionone

To construct bacteria which are capable to produce both delta carotene and AtCCD enzyme, E. coli BL21 CodonPlus-RIL (Stratagene) competent cells are transformed with pAC-Lyco-1 according to the manufacturer's instructions. Recombinant E. coli are selected overnight at 37° C. on LB-agar plates with 1% glucose and 30 ug/ml chloramphenicol. A colony of E. coli BL21 with pAC-Lyco-1 is inoculated in 1 ml LB with chloramphenicol and glucose and the culture is grown overnight at 250 rpm and 37° C. The BL21-pAC-Lyco-1 is made competent by diluting the overnight culture 100-fold in fresh LB medium with 1% glucose until, and shaking it at 37° C. until an optical density at 600 nm of 0.4 is reached. 10 ml of culture is centrifuged for 5 minutes at 400×g. Supernatant is discarded and replaced by 10 ml of an ice-cold solution of 10 mM CaCl₂ and 1 mM Tris-HCl pH=7.5. Cells are resuspended and immediately centrifuged again at 400×g for 5 minutes. After discarding the supernatant, cells are resuspended in 2 ml of an ice-cold solution of 75 mM CaCl₂ and 1 mM Tris-HCl pH=7.5. After incubation on ice for at least 30 minutes, cells are used for plasmid transformation by standard procedures. Plasmid pRSETA-RiCCD#1 (or pRSETA-AtCCD#1 or pRSETA-ZmCCD#1) is used to transform these cells, and transformed colonies are selected on LB-agar plates supplied with 1% glucose, 30 ug/ml chloramphenicol and 100 ug/ml ampicillin. The bacteria in the resulting colonies are referred to as BL21-pAC-Lyco-pRSETA-RiCCD.

4.7 Production of Pseudo Ionone

Production of pseudo ionone using BL21-pAC-Lyco-pRSETA-RiCCD bacteria is performed as described in section 1.6. Analysis of the products is performed as described in section 1.7. The strain has produced about 30 ug pseudo-ionone per 50 ml of culture.

4.8 Breakdown of Astaxanthin in Phaffia rhodozyma

To verify the ability of RiCCD to breakdown astaxanthin in Vivo, RiCCD is inserted into Phaffia rhodozyma. Generally, vectors capable of replicating in Saccharomyces cerevisiae also replicate in Phaffia rhodozyma. The pYES6/CT vector, purchased by InVitrogen, Carlsbad, Calif., USA, is used for this purpose. The RiCCD gene (SEQ ID NO:3) is added a GAATTCAATA DNA sequence before the N-terminal ATG start codon and a GAATTC after the C-terminal TAA stop codon by a conventional PCR method. This provides the necessary EcoRI restriction sites for cloning into the EcoRI site of pYES6/CT, and the AATA motif adjacent to the start codon to enhance transcription. The vector with the inserted RiCCD is amplified by transformation into E. coli and checked by sequencing to ensure the absence of PCR errors. The vector construct is transformed into Phaffia rhodozyme by the method described by Wery et al. (Wery, J., J. C. Verdoes, and A. J. J. van Ooyen. 1998. Efficient transformation of the astaxanthin-producing yeast Phaffia rhodozyma. Biotechnol. Technol. 12:339-405.), and isolated transformants obtained by plating the transformed cells onto blasticidin containing YPD plates containing. The efficiency of the RiCCD gene inserted in the transformants to reduce the astaxanthin content in Phaffia rhodozyma is very clear judged by the effect of organism coloration. The coloration is changed from bright orange to pale yellow. Comparing astaxanthin in wild type Phaffia Rhodozyme and in the RiCCD transformants is done by quantizing astaxanthin content by the procedure described by Johnson et al (E. A. Johnson et al (1978), App. Env. Microbiology Vol. 35, no. 6 p. 1155-1159).

Oligonucieotides

| | | |
|---|---|---|
| 2255 | PolyT | 5′TTTTTTTTTTTTTTTVV |
| | SP6 | 5′ATTTAGGTGACACTATA |
| | T7 | 5′AATACGACTCACTATAG |
| | PRSETrev | 5′TAGTTATTGCTCAGCGGTGG |
| | NUP | 5′AAGCAGTGGTATCAACGCAGAGT |
| 2260 | SmartIIA | 5′AAGCAGTGGTATCAACGCAGAGTACGCggg |
| | UPM Long | 5′CTATTACGACTCACTATAGGGCAAGCAGTGGTA |
| | | TCAACGCAGAGT |
| | UPM Short | 5′CTAATACGACTCACTATAGGGC |
| | 3′CDS | 5′AAGCAGTGGTATCAACGCAGAGTAC (T) |
| | | 30VN |
| 2265 | AtCCDanti | 5′GAATTCTTATATAAGAGTTTGTTCCTGGAG |
| | AtCCDsens | 5′GGATCCATGGCGGAGAAACTCAGTG |
| | AtCCDs791 | 5′CAGAGCCTATCATGATGCATG |
| | AtCCDa895 | 5′TTGTGGGATCAAATGATATCAT |
| | CCDfwd1 | 5′TGYYTIAAYGGIGARTTYGTIMGIGTIGGICCI |
| | | AAYCC |
| 2270 | CCDfwd2 | 5′GCIGGITAYCAYTGGTTYGAYGGIGAYGGIATG |
| | | ATHCAYGG |
| | CCDrev1 | 5′YTCIGTDATIGCRAARTCRTGCATCAT |
| | CCDrev2 | 5′GCRTAICKIGGIARIACICCRAAICKIGCYTTY |
| | | TTIGT |
| | RiCCDfwd1 | 5′CACTGGCGAGATGTTTACATTTG |
| | RiCCDfwd2 | 5′CAAAGGATGGTTTCATGCATGA |
| 2275 | RiCCDrev1 | 5′CTGCTGCATGTTAACCATGAGTAA |
| | RiCCDrev2 | 5′GATCTTCATAAATTTGGCACCTCC |
| | RiCCDstart | 5′ATATGGATCCATGGCAGAGGTCGCCGAG |
| | RiCCDend | 5′ATATGGTACCTTAGAGCCTTGCTTGTTCTTGCA |
| | RiCCDint1 | 5′TGAGCTGTATGAGATGAGATTCAAC |
| 2280 | RiCCDint2 | 5′GAAGTAATGCCAGGAACACG |
| | ZmCCDfwd1 | 5′GTCATCTCGCCGCAACC |
| | ZmCCDfwd2 | 5′CGCAGGATCCATGGGGACGGAGG |
| | ZmCCDrev1 | 5′CTGCAATAATTTCCAACCTGTAC |
| | ZmCCDrev2 | 5′ATATGAATTCAGGTGCCGTATCTTCACTGC |
| 2285 | ZmCCDint1 | 5′GACAAACTCATCAGATGGTTTCAAC |
| | ZmCCDint2 | 5′ATGAAGATTGGAGACCTTTGG |
| | CrtYmutS | 5′GGAACCATACCTGATTATTCCATGTGACGATCT |
| | | G |
| | CrtYmutA | 5′CAGATCGTCTTCATGGTTTATCAGGTATGGTTC |
| | | C |
| | CrtYseq | 5′ATCGTGAGGGATCTGATTTTAGTC |
| 2290 | CrtLfwd | 5′CGGGATCCAATGGAGTGTGTTGTGTTC |
| | CrtLrev | 5′ACGCGTCGACGAGGAGTTTACTCTTJTTG |
| | Dan3 | 5′AATGGAGTGTGTTGGAGTTC |
| | Dan4 | 5′CTGAAGTAATGAGGCCTCTG |
| | Dan5 | 5′TTCCCTGCAGGTTTGTGACT |
| 2295 | DanE | 5′ACAAACCTGCAGGGTTCAC |

V = (A, C or G)
Y = (C or T)
R = (A or G)
H = (A, C or T)
W = (A or T)
M = (A or C)
D = (A, G or T)
S = (C or G)
I = inosine
g = ribonucleotide G

Sequence Information

DNA sequences comprise the coding region of the cDNA of the indicated genes. Protein sequences comprise the amino acid sequence of the encoded protein

>AtCCD DNA

(SEQ ID NO:1)

ATGGCGGAGATCTCACTGATGGCAGCATCATCATCTCAGTCCATCCTAGA
CCCTCCAAGGGTTTCTCCTCGAAGCTTCTCGATCTTCTCGAGAGACTTGT
TGTCTGCTCATGCACGATGCTTCTCTCCCTCTCCACTACCTCTCAGGCTC
TTCGCTCCCATCCGTGATGAACTCCTCCCGTCATGGATCTCCCCGTCCAT
GGATTTCTTCCCGTTGCTTGTTGGTGAATTTGTGAGGGTTGGTCCTCCCC
TGTTTGATGCTGTCGCTGGATATCACTGGTTTGATGGAGATGGGATGATT
CATGGGGTACGCATCTAGATGGGTTCCTACTTATGTTTCTCGATATGTTA
GACATCACGTCTTTGCAGGTGAGTTCTTCGGAGCTGCCAATTCATGAAGA
TTGGTGACCTTTGGGGTTTTTCGGATTGCTTTGGTCTTTGCCAATACAGC
ACTCGTATATCACCATGATCTTCTAGCATTACAGGAGGCTTAAGCCGTAC
GTCATCATTTTTGGGTGAGGGAGACCTGCTCTCTGGGTATTTTAGATTAT
GACAAGAGATTGACCCACTCCTTCACTGCTCACCCTTGTTGACCCGGTTA
CGGGTGAAATGTTTACATTCGGCTATTCGCATACGCCACCTTATCTCACA
TACAGAGTTATCTCGATAAGATGGCATTATGCATGACCCCGTCCCTTTAC
TATATCAGAGCCTATCATGATGCATGATTTTGCTATTACTGAGACTTATG
CTTCTTCATGGATCTTCCTTCCCACAAAATGGCTCGTTTTGGTGTTCTTC
CACGCTATGCCTGGATGTCTTATGATTAGATGGTTTGAGCTTCCCTCTGC
TTTATTTTCCACTCGCCTTGCTTGGGTGTGAGGATGTGTCGTCCTCATCA
CTTGTCGTCTTGAGTTCCAGATCTTGAGTTGAGATTCAACATGACGGGCT
CAGCTTCTCATTTTT1TCTATCCGCACCTGCGGTTGATTTCCCCAGAATC
ATTTGAGTGCTACACCGGTGTAACAGAGATACGTATATGGAACAATTCTG
GACAGTATCGCTGGTTACCGGTTCATCTGTTTGATCTGCATGCAGACCTG
GGAGAAGGCAGATATGGTTCAGAGGCTATCTATGTTCCGCGTGAGACAGC
AGAAGAAGACGACGGTTACTTGATATTCTTTGTTCATGATGTCACAGGGT
TCACTGCCGCACAGGGTCCCATATGGCTTCCATGCCTTGTTTGTTACAGA
GGTCTCTCCAGGAACAATCTCTTATATTTTT
>AtCCD protein

(SEG ID NO:2)

MAEKITSDGSIIISVHPRPSKGFSSKLLDLLERLVVKTTHDASLPLHYLS
GNFAPIRDTPKFDAVAGYHWFDGDGMIHGVRIKDGTTYVSRYKTSRLKGE
EFFGTTTTGDLKGFFGLLMVNIGGLRTKITKILDNTYGNGTANTALVYHH
GKLLALGEANKPYVIKTQEEGDLGTLGIIDYDKRLITHSFTAHPKVDPVT
GEMFTFGYSHTPPYLTYRVISKDGIMHDPVPITTSEPIIVIMHDFAITET
YAIFMDLPMHFRPKENVKEKKMIYSFDPTKKARFGVLPRYAKDELMIRWF
ELPNCFIFHNTAWEEEDEVVLITCRLENPDLDNVSGKKEKLENFGNELYE
MRTMTGSASGKKLSAPAVTIKFDLTHAEAETGKRMLEVGGNIKGIYDLGE
GRYGSEAIYVPRETAEEDDGYLIFFVHDENTGKSCVTVIDAKTMSAEPVA
VVELPHRVPYGFWTTLFVTEEGLGEGTLI
>RiCCD DNA

(SEG ID NO:3)

ATGGCAGAGGTCGCCGAGTCAGCTTCACTTGACCCGTGCAGCATCAGCAG
CGGCTGGCCTCCMGGTGGTGGACTTGGTAGATGCTGATAGTCTGTTGATG
TACGACACTTCTCAGCCTCACCACTATCTCGCCGGTCTTCGCTCCGGTTG
TTGACGTCGCCTCCCACCTGGACCTCTCGTCATCGGCCATCTTCCTGATT
GCTTGTTGGAGAGTTCGTTAGGGTTGGCCCTCCCCTTTGTTTGCCCCAGT
TGCTGGATATCACTGGTTTGATGGAGATGGTATGATTCATGGTATGCGCA
TCTGATGGTGCTCATATGTCTTTATGAAGATCGGAGACCTTTGGGGCTTT
TTGGTTTACTCATGGTTTCATGCAGCAGCTGAGAGCTATCTGTATATTAG
ATATTTCATATGTTTGGGACAGGTTCACAGCTCTCATATATCACCATGGG
TCTTCTAGCTCTTTCAGAGGGATTTCCTTATGTTCTCAGTTCTGTGATGT
GATCTTCTCAGTTGGCTTGTTGGATTATGACAAGAGATTGMGCATTCCTT
CACTGCTCATCCTGGTTGACCCTTTCACTGGCGAGATGTTTACATTTGGC
TATTCGCATTTCCACCATATGTCACATACAGAGTTATTTCAAAGGATGGT
TTCATGTTGATCCTATACCCATTACAGTAGCAGCTCCTGTCATGATGCAT
GATTTTGCCATTACTGAGTCTATGCTTTTTCATGTTCTCCCCTTGTATAA
AAATGCTCGCTTTGGTGTCCTTCCTCGTTATGCTGGATTGTTGCTTTCAG
ATGGTTTGAGCTTCCAATTGCTTCATTTTCCATTTGCTTGCTTGGGAGGT
TGGACGAGTTGTTTTGATCACTTGCCGCCTTCTTCCAGATTTGGATATGG
TCTTGGGCCTGTCAAGMGCTTGTTTTCATATTTGAGCTGTATGAGATGAG
ATTCCCCAGGGTGTTGAGAGCTACACTGGCAGGTGCAACGCTATGTGTAT
GTCCACTGAGGTGGGACGATTGAGGTTGTGGA1TTGTCCAAGGCCTTTAT
GACCTGGGACCTGGTAGATTTGGTTCTGAGGCTTTTTTGTCCCTCGTGTT
CCTGGTTTACTTCAGAAGAAGATGATGGCTACTTTTATTCTTTGTACATT
TGAGTTACTGGATTCAGCAATACATGTACTAGATGCTTCTTGTCCAdTTT
CCCGTTGCAGTAGTTGAGTTGCCCCATAGAGTTCCATATGGGTTTCATGC
CTTCTTTGTGACAGAGGAGCT
>RiCCD protein

(SEG ID NO:4)

MAEVAEKGLHDDPKGHGGRHGSESNDPNPKPSGGTSTDLTTTTVKITMYD
TSGPHHYLAGNFAPVVDETPPTKDLNVIGHLPDCLNGEFVRVGPNPKFAP
VAGYHWFDGDGAAIHGAARIKDGKATYVSRYVKTSREKGEEYFGGTETTG
DLKGLFGLLMTMGGLRAKLKILDISYGIGTGNTALIYHHGKLLTSEGDKP
YTTEDGDLGTVGLLDYDKKHSFTAHPKVDPFTGEMTFGYSHNPPTTYRVI
SKDGTHDPIPITVMPVMMHDFAITENYAIFMDLPLYFRPKENVKEGKTTT
FSFDETKNARFGVLPRYCDELLIRWFELPNCFITNLKSGLATGKKLSESA
VDFPRXTTESYTGRKGRYVYGTTLDSIAKVTGIVKFDLHMPEVGKTKIEV
GGNVGGLYDLGPGRFGSEAITPRVPGITSEEDDGYLIFTHDENTGKSAIH
VLDAKTMSTDPVAWELPHRVPYGFTFTTEEQLQEQTT
>ZmCCD DNA

(SEG ID NO:5)

ATGGGGACGGAGGCGGGGCAGCCGGACATGGGCAGCCACCTTTCGACGGC
GTCGTGGTGGTGCCAGCGCCGCTCCCGCGTTTTTGGGGCTCGCCTCCTGG
GCGCTCGACCTGCTCGAGTCCCTCGCCGTGCGCCTCGGCCACGACTGACC
TGCCGCTCCACTGGCTCTCCGGCAACTTCGCCCCCGTCGTCGAGTGACCC
CGCCGGCCCCTACCTTACCGTCCGCGGACACCTCCCGGAGTGCTTGTTGG
AGAGTTTGTCAGGGTTGGGCCTTTCCGTGTTTGCTCCTGTTGCGGGGTAT
CACTGGTTTGATGGATCGGGATGATTCATGCCATGCGTATTAAGGATGGA
TGCTACCTATGTATCTGATATGTGTGACTGCCCGCCTCAAACAAGAGGAG
TATTTTGGTGGAGCGTTTATGTGATTGGAGACCTTTGGGATTTTTTGGAT
TGTTTATGGTCCTTGCAGCTCTTCGGTTTCTGTCTTGGATTTTACCTATG
GATTTGGGACAGCTTTACTGCACTTATATATCATTTGGTATGACTTGCAG
ACTCTTGGCTTGTTGGATTATGACTGGTTGTCATTCTTTTACTGCCCATC
CAGGTTGACCCTTTTACATTTTTGTTCACATTCGGATATTCACATGAACC
TCCATACTGTACATACCGTGTGATTTCTGACGTGCTATGCTTGATCCTTT
ACTCTATTTTTATGGACCTCCCTTTATTGTTCCGACCTGTGGTTAGTTTC
GGTGAGTTTATCTACTGTTTTTCCTACTGATGGTCGTTTTGGTATTCTCC
CCCGCTATGCATGGATGACCTCATCATTGGTTTCTCTCCCTTTTGTTTCA
TATTCGGGATTGAGCTGTACGAGATGAGATTCTCATGCTGTGCTGCTTCA
CTAGCAATTGTCTGTTTCTGCTGTGGATTTTCCTCGTGTTTTGATGCTAT
ACTGGCAGAAGCAGCGGTATGTCTACTGTCTATACTTGACAGCATTGCTG
GTGACTGGCGGAGGAAATGTACAAGGCATATATGACCTGGGACCTGGTAT
TTTGGTTCAGAGGCGATTTTTGTTCCCAGCATCCAGGTGTGTCCGGATGT
TTGACGTTATTTGATAACAATGTCTGCTGATCCAGTTGCGGTGGTTGAGC
TTCCTTTAGGGTTCCTTATGGATTCCATGCCTTCTTTGTATCTGAGGACC
TCTGGCTCGACAGGCGGAGGGGCAGTGA
>ZmCCD Protein

(SEG ID NO:6)
MGTEAGGPDMGSHRNDGPAPLPPKGTSWALDLLESLTGHDTKPLHTTSGN
FAPVVEETPPAPNLTVRGHLFECLNGEFTVGPNKFAPVAGYHWFDGDGMI
HAMRIKDGKATYVSRYVKTALKGEEYFGGKIGDLKGFFGLETGAAGGLTK
FKTDFTYGFGTANTALIYHHGKTTTSEADKPYTTTEDGDLGTLGLLDYDT
SFTAHPKVDPFTDEMFTFGYSHEPPYCTYRVINKDGTLDPVPITTPESWT
ITDFAITENYSIFMDLPITLFRPKEMTGEFIYKFDPTKKGRFGILPRYTT
DDTTTRWFGLPNCFIFHNANAWEEGDEVVTTTTCRLENPDLDKVNGYGSD
KLENFGNELYEMRFNMKTGTTTSGKGLSVSAVDFPRVNESYTGRKGRYVY
CTILDSIAKVTGIIKFDLHAEPESGVKELEVGGNVGGIYDLGPGRFGSTT
FVPKHPGVSGEEDDGYLIFFVHDENTGKSEVNVIDAKTMSADPVAVVELP
NRVPUGFHAFFVTEDQLARQAEGQ
>crtY DNA Sequence from PAC-Beta
(SEG ID NO:7)

GTGAGGGATCTGATTTTAGTCGGCGGCGGCCTGGCCTCGGGCTGATCGCC
TGGCGTCTGCGCCAGCGCTACCCGCAGCTTTTCCTGCTGCTGATCGAGGC
CGGGGAGCAGCCCGGCGGGTCCATACCTGGTCATTCCATTAGACGATCTT
CTCCCGGGCAGCACGCTGGCTGGCCCCGCTGGTTGCCGTCGCCTGGCCGG
GCTATGAGGTGCAGTTTCCCGATCTTCGCCGTCGCCTCGCGCGCGGCTAC
TACTCCATTACCTCAGAGCGCTTTGCCGAGGCCCTGCATCAGGCGCTGGG
GGAGTTCATCTGGCTWCTGTTCGGTGAGCGAGGTGTTACCCAATAGCGTG
CGCCTTGCCTTCGGTGAGGCGCTGCTTGCCGGAGCGGTGATGACGGACGC
GGCGTGACCGCCAGTTCGGCGATGCTTCCGGCTATCAGCTCTTTCTTGGT
CAGCAGTGGCGGCTGACACAGCCCCACGGCCTGACCGTACCTTCCTTTGG
ATGCCACGGTGGCGCAGCAGCAGGGCTATCGCTTTGTCTACACGCTGCCG
CTCTCCGCCGACACGCTGCTGATCGAGGATACGCGCTACGCC1TTGTCCC
GCAGCGTGATGATTTGCCCTACGCCAGACGGTTACCGACTATGCTCACAT
CTTTGGGTGGCAGCTGGCCCACTTGAACGCGAGGAGACCGGCTGTCTGCC
GATTACCCTGGCGGGTGACATCCAGGCTCTGTGGGCCGATGCGCCGGGCG
TGCCGCGCTCGGGTTGCGGGCTGGGCTATTTCACCCTACCACTGGCTATT
CGCTGCCGCTGGCGGTGGCCCTTGCCGACGCGATTGCCTCAGCCCGCGGC
TGGGCAGCGTTCCGCTCTATTGCTCACCCGGCAGTTTGCCGTCGCCACTG
GCGCAGGCAGGGATTCTTCCGCCTGCTGTCCGGATGCTTTTCCTGGCCGG
GCGCGAGGAGTCCGCTGGCGGGTGATGCAGCGCTTTTATGGGCTTCGTGC
CCACCGTAGAGCGCTTTTACGCCGGTCGGCTCTCTCTCTTTGATTGGCCC
GCATTTTGACGGGCAAGCCACCGGTTCCGCTGGGCGTGCCTGGCGGGCGG
CGCTGTCCATTTTCCTGACAGACGAGATATGGATGA
22CrtL-e DNA Y14387

(SEG ID NO:8)

ATGGAGTGTGTTGGAGTTCTGTTGGAGCTTGGCAGTTTTTCGCGTCCGAG
ATTGAACCGTTGGTCGGGAGGAGAGTTATGCCPTGTTGCATCTTTTTGGC
GTATGAGCAGTATGAAAGTAAATGTAATAGCAGTAGTGGTAGTGACAGTT
GTGTAGTTGATTAAAGAAGATTTTGCTGATGAAGAAGATTATATTTGCCG
GTGGTTCGCTCTTGTATTTGTTCWTGCAGCAGATAAAGATATGGATCAGC
AGTCTTGCTTTCTGATGAGTTACGACAAATATCTGCTGGACATTTTCCGT
ACTGGATTTAGTGGTTTCGGCTGTGGTCCTGCTGGTCTTGCTCTTGCCGC
GGAGTCAGCTTTGGTTTGTTTCGTGGGGCTCGTTGGGCCTGATCTTCCTT
TCACTCTCTATGGTGTATGGGAGTCGAGTTCTGATCTTGGTCTTCAAGCC
TGCATTGTCATGTTTGGCGGGATACCATTGTATATCTTGATGATGATGAA
CCTATTCTTATTGGCCGTGCCTATGGTGAGTTAGTCGCCATTTTCTGCAC
GAGGAGTTACTCAAMGGTGTGTGGAGGCAGGTGTTTTGTATCTTCTCGAA
AGTGGATAGGATTGTTGAGGCCACWTGGCCAGAGTCTTGTAGAGTGCGTG
GTGATGTTGTGATTCCCTGCAGGTTTGTGACTGTTGCATCGGGGGCAGCC
TCGGGGTTTCTTGCAGTATGAGTTGGGTTTGTCCTAGAGTTTCTGTTCTC
AGCTTATGGAGTGGAAGTTGAGGTTGATAACTTCCATTTGACCCGAGCCT
GATGGTTTTCATGGATTATAGAGATTATCTCAGACACGACGCTCTTCTTT
AGTGCTTTTATCCTCATTTCTTTATGCCATGCCCATGTCTCCAACACGAG
TCTTTTTCGAGTTTCTTGTTTGGCTTCAAAAGATGCAATGCCATTCGATC
TGTTTGTCTGATGCTACGATTGTTTCACCCTTGGTGTAAGATTAAGAAAT
TTACGAGGAGGTTGGTCTTACATACCGGTTGGTGGATCTTTGCCATACAG
TCTTTCACTTGCATTTGGTGCTGCTGCTAGCATGGTTCATCCAGCCACAG
GTTATTCAGTCGTCATTCACTTTCTGTGCTCCTTTTGCGCCTCTGTACTT
GCATATATTACGACTCATTATAGCAAGTCATGCTTACCAGTAAACGACAA
AGATCGTTTTTCCTATTTGGACTGGCTCTGATATTGCAGCTGGATATTGA
GGGGATAAGGTCATTTTTCCGCGCATTCTTCCGTGTGCCTTGTTGTGGCA
GGGATTTCTTGGTTCAAGTCTTTCTTCAGCATCCTCATGTTATTTGCCTT
CTACATGTTTATTATTGCACCAATTGACATGAGTTAGGCTTGATCAGACA
TCTTTTATCTGATCCTACTGGTGCTCATTGATTGTCTTATCTTACATTTT
AG
CrtL-e DNA

(SEG ID NO:9)

ATGGAGTGTGTTGGAGTTCAAMTGTTGGAGCTTGGCAGTTTTTACGCGTC
CGAGATTGAAACCGTTGGTCGGGAGGAGAGTTATGCCTTAGCTTCTTTTT
GGCGTATGAGCAGTATGAAAGTAATGTAATAGCAGTAGTGGTAGTGATGT
TGTGTAGTTGATTTGTTCAAATGCAGCAGTGATATGGATCAGCAGTCTTT
TGCTTTCTGATGAGTTGGCCTGATCTTCCTTTCACACTCTATGGTGTATG
GTGGACGAGTTCTTTCTTGGTCTTCAAGCCTGCATTGAACATGTTTGGCG
GGATACGTTTGTATATCTTTTGATGATGAACCTATTCTTATTGGCCGTGC
CTATGGTGAGTTAGTCGCCATTTTCTGCACGACGAGTTACTCAATAGGTG
TGTGGAGGCAGGTGTTTTGTATCTTCTCGTGTGGATAGGATTGTTGAGGC
CACAAATGGCCAGAGTCTTGTAGAGTGCGAGGGTGATGTTGTGATTCCCT
GCAGGTTTGTGACTGTTGCATCGGGGGCAGCCTCGGGGAAATTCTTGCAG
TATGAGTTGGGAGGTCCTAGAGTTTCTGTTCAAACAGCTTATGGAGTGGA
AGTTGAGGTTGATAACAATCCATTTGACCCGAGCCTGATGGTTTTCATGG
ATTATAGAGATTATGTCAGACACGACGCTCAATCTTTAGAAGCTAAATAT
CCAACATTTCTTTATGCCATGCCCATGTCTCCAACACGAGTCTTTTTCGA
GGAAACTTGTTTGGCTTCAAMGATGCAATGCCATTCGATCTGTTAAAGAA
AAAACTGATGCTACGATTGAACACCCTTGGTGTAAGAATTAAAGAAATTT
ACGAGGAGGAATGGTCTTACATACCGGTTGGTGGATCTTTGCCAAATACA
GAACAAAAAACACTTGCATTTGGTGCTGCTGCTAGCATGGTTCATCCAGC
CACAGGTTATTCAGTCGTCAGATCACTTTCTGAAGCTCCAAMTGCGCCTC
TGTACTTGCAAATATATTACGACAACATTATAGCAAGAACATGCTTACCA
GTTCAAGTATCCCGAGTATATCAACTCAAGCTTGGAACACTCTTTGGCCA
CATAGAACGAAAACGACAAAGATCGTTTTTCCTATTTGGACTGGCTCTGA
TATTGCAGCTGGATATTGAGGGGATAAGGTCATTTTTCCGCGCATTCTTC
CGTGTGCCAAAATGGATGTGGCAGGGATTTCTTGGTTCAAGTCTTTCTTC
AGCAGACCTCATGTTATTTGCCTTCTACATGTTTATTATTGCACCAAATG
ACATGAGAAAAGGCTTGATCAGACATCTTTTATCTGATCCTACTGGTGCA
ACATTGATAAGAACTTATCTTACATTTTAG
CrtL-e Protein

(SEG ID NO:10)

MECVGVGNVGAAAAVFTRPRLKPLVGRRVMPRKXGSFWRNSSMIVKCNSS
SGSDSCVVDKEDFADEEDYIKAGGSGLVFVGAAGGKKDMDGGSKLSDELR
GISAGGTVLDLVVIGCGPAGLALMESAKLGLWVGLVGPDLPFTNNYGVWE
DEFKDLGLGACIEHVWRDTIVYLDDDEPILIGRAYGRVSRHFLHEELLKR
CVEAGVLYLNSKVDRIVEATNGGSLVECEGDVVIPCRFVTVASGAASGKF
LGYELGGPRVSVGTAYGVEVEVDNNPFDPSLMVFMDYRDYVRHDAGSLEA
KYPTFTTYAAAPMSETRVFFEETCLASKDAAAPFDLLKKKLMLRLNTLGV
RIKEIYEEEWSYIPVGGSITPNTEGKTLAFGAAASMVHPATGYSVVRSLS
EAPKCASVLANILRGHYSKNMITTSSSIPSISTGAWNTLWPGERKRGRSF
FLFGLALILGLDEGIRSFFRAFFRVPKWMWGGFLGSSLSSADLMLFAFYM
FIIAPTTDMRKGLIRHLLSDPTGATLIRTYLTF

All publications mentioned in the above specification, and references cited in said publications, are herein incorporated by reference. Various modifications and variations of the described methods and system of the present invention will be apparent to those skilled in the art without departing from the scope and spirit of the present invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A host cell transformed or transfected with a nucleic acid encoding a plant-derived CCD enzyme.

2. A host cell as claimed in claim 1 wherein said plant-derived CCD enzyme comprises an amino acid sequence, or functional fragment thereof, as set out in any of SEQ ID NOS: 2, 4 or 6 or a sequence that is at least 75% homologous thereto.

3. A host cell as claimed in claim 1 or claim 2 which produces a carotenoid cleavage compound.

4. A host cell as claimed in any of claims 1 to 3 wherein the CCD enzyme is derived from Arabidopsis thaliana, Zea mais, tomato or Rubus idaeus.

5. A host cell as claimed in any of claims 1 to 4 wherein the CCD enzyme is derived from Rubus idaeus.

6. A host cell as claimed in any of claims 1 to 5 wherein said host cell is a microorganism.

7. A host cell as claimed in claim 6 wherein said microorganism is a eukaryotic cell and, preferably, fungi.

8. A host cell as claimed in claim 7 wherein said microorganism is Phaffia rhodozyma.

9. A host cell as claimed in claim 6 wherein said microorganism is a prokaryotic bacterial cell and, preferably, E. coli.

10. A host cell as claimed in any of claims 1 to 9 wherein said host cell produces at least one of GGDP, β carotene, lycopene or delta carotene.

11. A host cell as claimed in any of claims 1 to 10 wherein said host cell is additionally transformed or transfected with an additional nucleic acid encoding a carotenoid-biosynthesis enzyme.

12. A host cell as claimed in claim 11 wherein said additional nucleic acid encodes one or more enzymes selected from the group consisting of i) an enzyme that converts geranylgeranyl diphosphate to β carotene, ii) crtB and iii) crtL-e.

13. A host cell as claimed in claim 12 wherein said additional nucleic acid has a sequence as set out in SEQ ID NO: 9.

14. A plasmid or vector system comprising a nucleotide sequence as set out in any of SEQ ID Nos: 1, 3, 5 or 9 or a sequence that is at least 75% homologous thereto.

15. A method of producing a carotenoid cleavage compound comprising treating a carotenoid with a plant-derived carotenoid cleavage dioxygenase (CCD).

16. A method as claimed in claim 15 wherein the CCD comprises an amino acid sequence set out in SEQ ID NOs: 2, 4, 6 or 10 or a sequence having at least 75% homology thereto or an effective fragment thereof.

17. A method for producing a carotenoid cleavage compound which comprises:
a) providing a host cell that produces a carotenoid wherein the host cell comprises at least one expressible transgene comprising a plant-derived CCD;
b) culturing the transgenic organism under conditions suitable for expression of a transgene; and
c) recovering the carotenoid cleavage compound from the culture.

18. A method as claimed in claim 17 wherein said host cell further comprises an expressible transgene comprising a second carotenoid biosynthetic enzyme.

19. A method as claimed in claim 18 wherein the carotenoid biosynthetic enzyme is epsilon cyclase.

20. A method as claimed in any of claims 15 to 19 wherein the plant-derived CCD is selected from AtCCD, RiCCD or ZmCCD enzyme.

21. A method as claimed in any of claims 15 to 20 wherein the carotenoid is α, β, γ or δ carotene or lycopene.

22. A method as claimed in any of claims 15 to 21 wherein the carotenoid cleavage compound is α or β ionone, pseudo ionone, safranal, theaspirone, damascone or damascenone.

23. An enzyme comprising the amino acid sequence corresponding to Rubus idaeus CCD or a functional equivalent thereof or an effective fragment thereof.

24. An enzyme comprising the amino acid sequence as shown in SEQ ID NO: 4 or a sequence having at least 75% homology thereto or an effective fragment thereof.

25. An isolated nucleic acid molecule coding for the enzyme of claim 23 or claim 24.

26. An isolated nucleic acid molecule comprising a nucleotide sequence that is the same as, or is complementary to, or contains any suitable codon substitutions for any of those of SEQ ID NO: 3 or comprises a sequence which has at least 75% sequence homology with SEQ ID NO: 3.

27. An isolated nucleic acid molecule comprising the sequence as set out in SEQ ID NO: 3.

28. A CrtL-e enzyme comprising the amino acid sequence as shown in SEQ ID NO: 10 or an effective fragment thereof.

29. An isolated nucleic acid molecule coding for we enzyme CrtL-e enzyme having the sequence set out in SEQ ID NO: 9.


SEQUENCE LISTING (51 sequences)

SEQ ID NO: 1 - DNA, 1617 bp, Arabidopsis thaliana
atggcggagaaactcagtgatggcagcatcatcatctcagtccatcctagaccctccaagggtttctcctcgaagcttctcgatcttctcgagagacttgttgtcaagctcatgcacgatgcttctctccctctccactacctctcaggcaacttcgctcccatccgtgatgaaactcctcccgtcaaggatctccccgtccatggatttcttcccgaatgcttgaatggtgaatttgtgagggttggtccaaaccccaagtttgatgctgtcgctggatatcactggtttgatggagatgggatgattcatggggtacgcatcaaagatgggaaagctacttatgtttctcgatatgttaagacatcacgtcttaagcaggaagagttcttcggagctgccaaattcatgaagattggtgaccttaaggggtttttcggattgctaatggtcaatatccaacagctgagaacgaagctcaaaatattggacaacacttatggaaatggaactgccaatacagcactcgtatatcaccatggaaaacttctagcattacaggaggcaaataagccgtacgtcatcaaattttgggaagagggagacctgcaaactctgggtataatagattatgacaagagattgacccactccttcactgctcacccaaaagttgacccggttacgggtgaaatgtttacattcggctattcgcatacgccaccttatctcacatacagagttatctcgaaagatggcattatgcatgaccccgtcccaattactatatcagagcctatcatgatgcatgattttgctattactgagacttatgcaatcttcatggatcttcctatgcacttcaggccaaaggaaatggtgaaagagaagaaaatgatatactcatttgatcccacaaaaaaggctcgttttggtgttcttccacgctatgccaaggatgaacttatgattagatggtttgagcttcccaactgctttattttccacaacgccaatgcttgggaagaagaggatgaagtcgtcctcatcacttgtcgtcttgagaatccagatcttgacatggtcagtgggaaagtgaaagaaaaactcgaaaattttggcaacgaactgtacgaaatgagattcaacatgaaaacgggctcagcttctcaaaaaaaactatccgcacctgcggttgatttccccagaatcaatgagtgctacaccggaaagaaacagagatacgtatatggaacaattctggacagtatcgcaaaggttaccggaatcatcaagtttgatctgcatgcagaagctgagacagggaaaagaatgctggaagtaggaggtaatatcaaaggaatatatgacctgggagaaggcagatatggttcagaggctatctatgttccgcgtgagacagcagaagaagacgacggttacttgatattctttgttcatgatgaaaacacagggaaatcatgcgtgactgtgatagacgcaaaaacaatgtcggctgaaccggtggcagtggtggagctgccgcacagggtcccatatggcttccatgccttgtttgttacagaggaacaactccaggaacaaactcttatataa

SEQ ID NO: 2 - protein, 538 aa, Arabidopsis thaliana
MAEKLSDGSIIISVHPRPSKGFSSKLLDLLERLVVKLMHDASLPLHYLSGNFAPIRDETPPVKDLPVHGFLPECLNGEFVRVGPNPKFDAVAGYHWFDGDGMIHGVRIKDGKATYVSRYVKTSRLKQEEFFGAAKFMKIGDLKGFFGLLMVNIQQLRTKLKILDNTYGNGTANTALVYHHGKLLALQEANKPYVIKFWEEGDLQTLGIIDYDKRLTHSFTAHPKVDPVTGEMFTFGYSHTPPYLTYRVISKDGIMHDPVPITISEPIMMHDFAITETYAIFMDLPMHFRPKEMVKEKKMIYSFDPTKKARFGVLPRYAKDELMIRWFELPNCFIFHNANAWEEEDEVVLITCRLENPDLDMVSGKVKEKLENFGNELYEMRFNMKTGSASQKKLSAPAVDFPRINECYTGKKQRYVYGTILDSIAKVTGIIKFDLHAEAETGKRMLEVGGNIKGIYDLGEGRYGSEAIYVPRETAEEDDGYLIFFVHDENTGKSCVTVIDAKTMSAEPVAVVELPHRVPYGFHALFVTEEQLQEQTLI

SEQ ID NO: 3 - DNA, 1696 bp, Rubus idaeus
atggcagaggtcgccgagaaacagcttcacgatgacccgaagcagcatcagcagcggcatcagagcgagagcaacgacgtcgtagtggtgccgaatcccaagcccagcaagggcctggcctccaaggtggtggacttggtagagaagctgatagtcaagttgatgtacgacacttctcagcctcaccactatctcgccggaaacttcgctccggttgttgacgaaacgcctcccaccaaggacctcaacgtcatcggccatcttcctgattgcttgaatggagagttcgttagggttggcccaaaccccaagtttgccccagttgctggatatcactggtttgatggagatggtatgattcatggtatgcgcatcaaagatgggaaagcaacatatgtctcccgctatgtaaagacatcgcgttttaagcaagaagaatactttggaggtgccaaatttatgaagatcggagaccttaaggggctttttggtttactcatggttaacatgcagcagctgagagctaaactgaaaatattagatatttcatatggaattgggacaggtaacacagctctcatatatcaccatgggaaacttctagctctttcagagggagataaaccttatgttctcaaagttctggaagatggagatcttcaaacagttggcttgttggattatgacaagagattgaagcattccttcactgctcatccaaaggttgaccctttcactggcgagatgtttacatttggctattcgcataatccaccatatgtcacatacagagttatttcaaaggatggtttcatgcatgatcctatacccattacagtagcagctcctgtcatgatgcatgattttgccattactgagaactatgcaattttcatggatctccccttgtatttcagaccaaaggaaatggtgaaggaaggcaagctgatattctcatttgatgagaccaaaaatgctcgctttggtgtccttcctcgttatgcaaaggatgagttgctaatcagatggtttgagcttccaaattgcttcattttccataatgccaatgcttgggaggaagaggacgaagttgttttgatcacttgccgccttcaaaatccagatttggatatggtcaatgggcctgtcaagaaaaagcttgaaaatttcaaaaatgagctgtatgagatgagattcaacttaaaaagtggtctagcaacacagaagaaattatcagaatcagctgtagattttcccagggtgaatgagagctacactggcaggaagcaacgctatgtgtatggaaccactctggatagcattgcgaaggtgactgggatagtcaagtttgatcttcatgctgccccagaggtgggaaaaacgaaaattgaggttggaggaaatgtccaaggcctttatgacctgggacctggtagatttggttctgaggcaatttttgtccctcgtgttcctggcattacttcagaagaagatgatggctacttaatattctttgtacatgatgagaatactggaaaatcagcaatacatgtactagatgcaaaaacaatgtccactgatcccgttgcagtagttgagttgccccatagagttccatatgggtttcatgccttctttgtgacagaggagcaattgcaagaacaagcaaggctctaaggtaccatataatcactag

SEQ ID NO: 4 - protein, 558 aa, Rubus idaeus
MAEVAEKQLHDDPKQHQQRHQSESNDVVVVPNPKPSKGLASKVVDLVEKLIVKLMYDTSQPHHYLAGNFAPVVDETPPTKDLNVIGHLPDCLNGEFVRVGPNPKFAPVAGYHWFDGDGMIHGMRIKDGKATYVSRYVKTSRFKQEEYFGGAKFMKIGDLKGLFGLLMVNMQQLRAKLKILDISYGIGTGNTALIYHHGKLLALSEGDKPYVLKVLEDGDLQTVGLLDYDKRLKHSFTAHPKVDPFTGEMFTFGYSHNPPYVTYRVISKDGFMHDPIPITVAAPVMMHDFAITENYAIFMDLPLYFRPKEMVKEGKLIFSFDETKNARFGVLPRYAKDELLIRWFELPNCFIFHNANAWEEEDEVVLITCRLQNPDLDMVNGPVKKKLENFKNELYEMRFNLKSGLATQKKLSESAVDFPRVNESYTGRKQRYVYGTTLDSIAKVTGIVKFDLHAAPEVGKTKIEVGGNVQGLYDLGPGRFGSEAIFVPRVPGITSEEDDGYLIFFVHDENTGKSAIHVLDAKTMSTDPVAVVELPHRVPYGFHAFFVTEEQLQEQARL

SEQ ID NO: 5 - DNA, 1653 bp, Zea mais
atggggacggaggcggggcagccggacatgggcagccaccgaaacgacggcgtcgtggtggtgccagcgccgctcccgcgtaaggggctcgcctcctgggcgctcgacctgctcgagtccctcgccgtgcgcctcggccacgacaagaccaagccgctccactggctctccggcaacttcgcccccgtcgtcgaggagaccccgccggccccaaaccttaccgtccgcggacacctcccggagtgcttgaatggagagtttgtcagggttgggcctaatccgaagtttgctcctgttgcggggtatcactggtttgatggagacgggatgattcatgccatgcgtattaaggatggaaaagctacctatgtatcaagatatgtgaagactgcccgcctcaaacaagaggagtattttggtggagcaaagtttatgaagattggagaccttaagggattttttggattgtttatggtccaaatgcagcaacttcggaaaaaattcaaagtcttggattttacctatggatttgggacagctaatactgcacttatatatcatcatggtaaactcatggccttgtcagaagcagataagccatatgttgttaaggtccttgaagatggagacttgcagactcttggcttgttggattatgacaaaaggttgaaacattcttttactgcccatccaaaggttgacccttttacagatgaaatgttcacattcggatattcacatgaacctccatactgtacataccgtgtgattaacaaagacggagctatgcttgatcctgtgccaataacaataccggaatctgtaatgatgcatgattttgccatcacagagaattactctatttttatggacctccctttattgttccgaccaaaggaaatggtgaagaacggtgagtttatctacaagtttgatcctacaaagaaaggtcgttttggtattctcccccgctatgcaaaggatgacaaactcatcagatggtttcaactccctaattgtttcatattccataatgctaatgcttgggaagagggtgatgaagttgttctcattacctgccgccttgagaatccagatttggacaaggtgaatggatatcaaagtgacaagctcgaaaacttcgggaatgagctgtacgagatgagattcaacatgaaaacgggtgctgcttcacaaaagcaattgtctgtttctgctgtggattttcctcgtgttaatgagagctatactggcagaaagcagcggtatgtctactgcactatacttgacagcattgcgaaggtgactggcatcataaagtttgatctgcatgctgaaccggaaagtggtgtgaaagaacttgaagtgggaggaaatgtacaaggcatatatgacctgggacctggtagatttggttcagaggcgatttttgttcccaagcatccaggtgtgtccggagaagaagatgacggctatttgatattctttgtacacgacgagaatacagggaaatctgaagtaaatgttatcgatgcaaagacaatgtctgctgatccagttgcggtggttgagcttcctaatagggttccttatggattccatgccttctttgtaactgaggaccaactggctcgacaggcggaggggcagtga

SEQ ID NO: 6 - protein, 550 aa, Zea mais
MGTEAGQPDMGSHRNDGVVVVPAPLPRKGLASWALDLLESLAVRLGHDKTKPLHWLSGNFAPVVEETPPAPNLTVRGHLPECLNGEFVRVGPNPKFAPVAGYHWFDGDGMIHAMRIKDGKATYVSRYVKTARLKQEEYFGGAKFMKIGDLKGFFGLFMVQMQQLRKKFKVLDFTYGFGTANTALIYHHGKLMALSEADKPYVVKVLEDGDLQTLGLLDYDKRLKHSFTAHPKVDPFTDEMFTFGYSHEPPYCTYRVINKDGAMLDPVPITIPESVMMHDFAITENYSIFMDLPLLFRPKEMVKNGEFIYKFDPTKKGRFGILPRYAKDDKLIRWFQLPNCFIFHNANAWEEGDEVVLITCRLENPDLDKVNGYQSDKLENFGNELYEMRFNMKTGAASQKQLSVSAVDFPRVNESYTGRKQRYVYCTILDSIAKVTGIIKFDLHAEPESGVKELEVGGNVQGIYDLGPGRFGSEAIFVPKHPGVSGEEDDGYLIFFVHDENTGKSEVNVIDAKTMSADPVAVVELPNRVPYGFHAFFVTEDQLARQAEGQ

SEQ ID NO: 7 - DNA, 1161 bp, Artificial
  crtY DNA sequence from pAC-Beta
gtgagggatctgattttagtcggcggcggcctggccaacgggctgatcgcctggcgtctgcgccagcgctacccgcagcttaacctgctgctgatcgaggccggggagcagcccggcgggaaccatacctggtcattccatgaagacgatctgactcccgggcagcacgcctggctggccccgctggtggcccacgcctggccgggctatgaggtgcagtttcccgatcttcgccgtcgcctcgcgcgcggctactactccattacctcagagcgctttgccgaggccctgcatcaggcgctgggggagaacatctggctaaactgttcggtgagcgaggtgttacccaatagcgtgcgccttgccaacggtgaggcgctgcttgccggagcggtgattgacggacgcggcgtgaccgccagttcggcgatgcaaaccggctatcagctctttcttggtcagcagtggcggctgacacagccccacggcctgaccgtaccgatcctgatggatgccacggtggcgcagcagcagggctatcgctttgtctacacgctgccgctctccgccgacacgctgctgatcgaggatacgcgctacgccaatgtcccgcagcgtgatgataatgccctacgccagacggttaccgactatgctcacagcaaagggtggcagctggcccagcttgaacgcgaggagaccggctgtctgccgattaccctggcgggtgacatccaggctctgtgggccgatgcgccgggcgtgccgcgctcgggaatgcgggctgggctatttcaccctaccactggctattcgctgccgctggcggtggcccttgccgacgcgattgccgacagcccgcggctgggcagcgttccgctctatcagctcacccggcagtttgccgaacgccactggcgcaggcagggattcttccgcctgctgaaccggatgcttttcctggccgggcgcgaggagaaccgctggcgggtgatgcagcgcttttatgggctgccggagcccaccgtagagcgcttttacgccggtcggctctctctctttgataaggcccgcattttgacgggcaagccaccggttccgctgggcgaagcctggcgggcggcgctgaaccattttcctgacagacgagataaaggatga

SEQ ID NO: 8 - DNA, 1581 bp, Lycopersicon esculentum
atggagtgtgttggagttcaaaatgttggagcaatggcagttttaacgcgtccgagattgaaccgttggtcgggaggagagttatgccaagaaaaaagcatctttttggcgtatgagcagtatgaaagtaaatgtaatagcagtagtggtagtgacagttgtgtagttgataaagaagattttgctgatgaagaagattatataaaagccggtggttcgcaacttgtatttgttcaaatgcagcagaaaaaagatatggatcagcagtctaagctttctgatgagttacgacaaatatctgctggacaaaccgtactggatttagtggtaatcggctgtggtcctgctggtcttgctcttgccgcggagtcagctaaattggggttgaacgtggggctcgttgggcctgatcttcctttcacaaacaactatggtgtatgggaggacgagttcaaagatcttggtcttcaagcctgcattgaacatgtttggcgggataccattgtatatcttgatgatgatgaacctattcttattggccgtgcctatggaagagttagtcgccattttctgcacgaggagttactcaaaaggtgtgtggaggcaggtgttttgtatctaaactcgaaagtggataggattgttgaggccacaaatggccagagtcttgtagagtgcgaaggtgatgttgtgattccctgcaggtttgtgactgttgcatcgggggcagcctcggggaaattcttgcagtatgagttgggaagtcctagagtttctgttcaaacagcttatggagtggaagttgaggttgataacaatccatttgacccgagcctgatggttttcatggattatagagattatctcagacacgacgctcaatctttagaagctaaatatccaacatttctttatgccatgcccatgtctccaacacgagtctttttcgaggaaacttgtttggcttcaaaagatgcaatgccattcgatctgttaaagaaaaaactgatgctacgattgaacacccttggtgtaagaattaaagaaatttacgaggaggaatggtcttacataccggttggtggatctttgccaaatacagaacaaaaaacacttgcatttggtgctgctgctagcatggttcatccagccacaggttattcagtcgtcagatcactttctgaagctccaaaatgcgcctctgtacttgcaaatatattacgacaacattatagcaagaacatgcttaccagttcaagtatcccgagtatatcaactcaagcttggaacactctttggccacaagaacgaaaacgacaaagatcgtttttcctatttggactggctctgatattgcagctggatattgaggggataaggtcatttttccgcgcattcttccgtgtgccaaaatggatgtggcagggatttcttggttcaagtctttcttcagcagacctcatgttatttgccttctacatgtttattattgcaccaaatgacatgagaaaaggcttgatcagacatcttttatctgatcctactggtgcaacattgataagaacttatcttacattttag

SEQ ID NO: 9 - DNA, 1584 bp, Lycopersicon spp
atggagtgtgttggagttcaaaatgttggagcaatggcagtttttacgcgtccgagattgaaaccgttggtcgggaggagagttatgccaagaaaaaagcaatctttttggcgtatgagcagtatgaaagtaaaatgtaatagcagtagtggtagtgacagttgtgtagttgataaagaagattttgctgatgaagaagattatataaaagccggtggttcgcaacttgtatttgttcaaatgcagcagaaaaaagatatggatcagcagtctaagctttctgatgagttacgacaaatatctgctggacaaactgtactggatttagtggtaatcggctgtggtcctgctggtcttgctcttgccgcggagtcagctaaattggggttgaacgtggggctcgttgggcctgatcttcctttcacaaacaactatggtgtatgggaggacgagttcaaagatcttggtcttcaagcctgcattgaacatgtttggcgggataccattgtatatcttgatgatgatgaacctattcttattggccgtgcctatggaagagttagtcgccattttctgcacgaggagttactcaaaaggtgtgtggaggcaggtgttttgtatctaaactcgaaagtggataggattgttgaggccacaaatggccagagtcttgtagagtgcgagggtgatgttgtgattccctgcaggtttgtgactgttgcatcgggggcagcctcggggaaattcttgcagtatgagttgggaggtcctagagtttctgttcaaacagcttatggagtggaagttgaggttgataacaatccatttgacccgagcctgatggttttcatggattatagagattatgtcagacacgacgctcaatctttagaagctaaatatccaacatttctttatgccatgcccatgtctccaacacgagtctttttcgaggaaacttgtttggcttcaaaagatgcaatgccattcgatctgttaaagaaaaaactgatgctacgattgaacacccttggtgtaagaattaaagaaatttacgaggaggaatggtcttacataccggttggtggatctttgccaaatacagaacaaaaaacacttgcatttggtgctgctgctagcatggttcatccagccacaggttattcagtcgtcagatcactttctgaagctccaaaatgcgcctctgtacttgcaaatatattacgacaacattatagcaagaacatgcttaccagttcaagtatcccgagtatatcaactcaagcttggaacactctttggccacaagaacgaaaacgacaaagatcgtttttcctatttggactggctctgatattgcagctggatattgaggggataaggtcatttttccgcgcattcttccgtgtgccaaaatggatgtggcagggatttcttggttcaagtctttcttcagcagacctcatgttatttgccttctacatgtttattattgcaccaaatgacatgagaaaaggcttgatcagacatcttttatctgatcctactggtgcaacattgataagaacttatcttacattttag

SEQ ID NO: 10 - protein, 527 aa, Lycopersicon spp
MECVGVQNVGAMAVFTRPRLKPLVGRRVMPRKKQSFWRMSSMKVKCNSSSGSDSCVVDKEDFADEEDYIKAGGSQLVFVQMQQKKDMDQQSKLSDELRQISAGQTVLDLVVIGCGPAGLALAAESAKLGLNVGLVGPDLPFTNNYGVWEDEFKDLGLQACIEHVWRDTIVYLDDDEPILIGRAYGRVSRHFLHEELLKRCVEAGVLYLNSKVDRIVEATNGQSLVECEGDVVIPCRFVTVASGAASGKFLQYELGGPRVSVQTAYGVEVEVDNNPFDPSLMVFMDYRDYVRHDAQSLEAKYPTFLYAMPMSPTRVFFEETCLASKDAMPFDLLKKKLMLRLNTLGVRIKEIYEEEWSYIPVGGSLPNTEQKTLAFGAAASMVHPATGYSVVRSLSEAPKCASVLANILRQHYSKNMLTSSSIPSISTQAWNTLWPQERKRQRSFFLFGLALILQLDIEGIRSFFRAFFRVPKWMWQGFLGSSLSSADLMLFAFYMFIIAPNDMRKGLIRHLLSDPTGATLIRTYLTF

SEQ ID NO: 11 - DNA, 17 bp, Artificial
  Oligonucleotide primer
tttttttttttttttvv

SEQ ID NO: 12 - DNA, 17 bp, Artificial
  Oligonucleotide primer
atttaggtgacactata

SEQ ID NO: 13 - DNA, 17 bp, Artificial
  Oligonucleotide primer
aatacgactcactatag

SEQ ID NO: 14 - DNA, 20 bp, Artificial
  Oligonucleotide primer
tagttattgctcagcggtgg

SEQ ID NO: 15 - DNA, 23 bp, Artificial
  Oligonucleotide primer
aagcagtggtatcaacgcagagt

SEQ ID NO: 16 - DNA, 30 bp, Artificial
  Oligonucleotide primer
aagcagtggtatcaacgcagagtacgcggg

SEQ ID NO: 17 - DNA, 45 bp, Artificial
  Oligonucleotide primer
ctaatacgactcactatagggcaagcagtggtatcaacgcagagt

SEQ ID NO: 18 - DNA, 22 bp, Artificial
  Oligonucleotide primer
ctaatacgactcactatagggc

SEQ ID NO: 19 - DNA, 57 bp, Artificial
  Oligonucleotide primer
  misc_feature (57)..(57): n is a, c, g, or t
aagcagtggtatcaacgcagagtacttttttttttttttttttttttttttttttvn

SEQ ID NO: 20 - DNA, 30 bp, Artificial
  Oligonucleotide primer
gaattcttatataagagtttgttcctggag

SEQ ID NO: 21 - DNA, 25 bp, Artificial
  Oligonucleotide primer
ggatccatggcggagaaactcagtg

SEQ ID NO: 22 - DNA, 21 bp, Artificial
  Oligonucleotide primer
cagagcctatcatgatgcatg

SEQ ID NO: 23 - DNA, 22 bp, Artificial
  Oligonucleotide primer
ttgtgggatcaaatgatatcat

SEQ ID NO: 24 - DNA, 38 bp, Artificial
  Oligonucleotide primer
  misc_feature (6)..(6): n is the modified nucleotide inosine
  misc_feature (12)..(12): n is the modified nucleotide inosine
  misc_feature (21)..(21): n is the modified nucleotide inosine
  misc_feature (24)..(24): n is the modified nucleotide inosine
  misc_feature (27)..(27): n is the modified nucleotide inosine
  misc_feature (30)..(30): n is the modified nucleotide inosine
  misc_feature (33)..(33): n is the modified nucleotide inosine
tgyytnaayggngarttygtnmgngtnggnccnaaycc

SEQ ID NO: 25 - DNA, 41 bp, Artificial
  Oligonucleotide primer
  misc_feature (3)..(3): n is the modified nucleotide inosine.
  misc_feature (6)..(6): n is the modified nucleotide inosine.
  misc_feature (24)..(24): n is the modified nucleotide inosine.
  misc_feature (30)..(30): n is the modified nucleotide inosine.
gcnggntaycaytggttygayggngayggnatgathcaygg

SEQ ID NO: 26 - DNA, 27 bp, Artificial
  Oligonucleotide primer
  misc_feature (4)..(4): n is the modified nucleotide inosine.
  misc_feature (10)..(10): n is the modified nucleotide inosine.
ytcngtdatngcraartcrtgcatcat

SEQ ID NO: 27 - DNA, 38 bp, Artificial
  Oligonucleotide primer
  misc_feature (6)..(6): n is the modified nucleotide inosine.
  misc_feature (9)..(9): n is the modified nucleotide inosine.
  misc_feature (12)..(12): n is the modified nucleotide inosine.
  misc_feature (15)..(15): n is the modified nucleotide inosine.
  misc_feature (18)..(18): n is the modified nucleotide inosine.
  misc_feature (24)..(24): n is the modified nucleotide inosine.
  misc_feature (27)..(27): n is the modified nucleotide inosine.
  misc_feature (36)..(36): n is the modified nucleotide inosine.
gcrtancknggnarnacnccraanckngcyttyttngt

SEQ ID NO: 28 - DNA, 23 bp, Artificial
  Oligonucleotide primer
cactggcgagatgtttacatttg

SEQ ID NO: 29 - DNA, 22 bp, Artificial
  Oligonucleotide primer
caaaggatggtttcatgcatga

SEQ ID NO: 30 - DNA, 24 bp, Artificial
  Oligonucleotide primer
ctgctgcatgttaaccatgagtaa

SEQ ID NO: 31 - DNA, 24 bp, Artificial
  Oligonucleotide primer
gatcttcataaatttggcacctcc

SEQ ID NO: 32 - DNA, 28 bp, Artificial
  Oligonucleotide primer
atatggatccatggcagaggtcgccgag

SEQ ID NO: 33 - DNA, 33 bp, Artificial
  Oligonucleotide primer
atatggtaccttagagccttgcttgttcttgca

SEQ ID NO: 34 - DNA, 25 bp, Artificial
  Oligonucleotide primer
tgagctgtatgagatgagattcaac

SEQ ID NO: 35 - DNA, 20 bp, Artificial
  Oligonucleotide primer
gaagtaatgccaggaacacg

SEQ ID NO: 36 - DNA, 17 bp, Artificial
  Oligonucleotide primer
gtcatctcgccgcaacc

SEQ ID NO: 37 - DNA, 23 bp, Artificial
  Oligonucleotide primer
cgcaggatccatggggacggagg

SEQ ID NO: 38 - DNA, 23 bp, Artificial
  Oligonucleotide primer
ctgcaataatttccaacctgtac

SEQ ID NO: 39 - DNA, 30 bp, Artificial
  Oligonucleotide primer
atatgaattcaggtgccgtatcttcactgc

SEQ ID NO: 40 - DNA, 25 bp, Artificial
  Oligonucleotide primer
gacaaactcatcagatggtttcaac

SEQ ID NO: 41 - DNA, 22 bp, Artificial
  Oligonucleotide primer
atgaagattggagaccttaagg

SEQ ID NO: 42 - DNA, 35 bp, Artificial
  Oligonucleotide primer
ggaaccatacctgataattccatgaagacgatctg

SEQ ID NO: 43 - DNA, 35 bp, Artificial
  Oligonucleotide primer
cagatcgtcttcatggaattatcaggtatggttcc

SEQ ID NO: 44 - DNA, 24 bp, Artificial
  Oligonucleotide primer
atcgtgagggatctgattttagtc

SEQ ID NO: 45 - DNA, 28 bp, Artificial
  Oligonucleotide primer
cgggatccaatggagtgtgttggagttc

SEQ ID NO: 46 - DNA, 30 bp, Artificial
  Oligonucleotide primer
acgcgtcgacgaggagtttactctaaaatg

SEQ ID NO: 47 - DNA, 20 bp, Artificial
  Oligonucleotide primer
aatggagtgtgttggagttc

SEQ ID NO: 48 - DNA, 20 bp, Artificial
  Oligonucleotide primer
ctgaagtaatgaggcctctg

SEQ ID NO: 49 - DNA, 20 bp, Artificial
  Oligonucleotide primer
ttccctgcaggtttgtgact

SEQ ID NO: 50 - DNA, 20 bp, Artificial
  Oligonucleotide primer
acaaacctgcagggaatcac

SEQ ID NO: 51 - DNA, 10 bp, Artificial
  Sequence added for cloning purposes
gaattcaata